(19) United States
(12) Patent Application Publication
(10) Pub. No.: US 2002/0110867 A1
(43) Pub. Date: Aug. 15, 2002

(54) Cardiac and pancreatic protein and gene

(75) Inventors: Daniel R. Soppet, Centreville, VA (US); Steven M. Ruben, Olney, MD (US)

Correspondence Address: STERNE, KESSLER, GOLDSTEIN & FOX P.L.L.C., 1100 NEW YORK AVENUE, N.W., SUITE 600, WASHINGTON, DC, 20005-3934, US

(73) Assignee: Human Genome Sciences

(21) Appl. No.: 09/972,912

(22) Filed: Oct. 10, 2001

Related U.S. Application Data
(63) Continuation of application No. 09/049,022, filed on Mar. 27, 1998, now abandoned.
(60) Provisional application No. 60/042,855, filed on Mar. 28, 1997.

(51) Int. Cl. C07K 14/475; C07H 21/04; C12P 21/02; C12N 5/06

(52) U.S. Cl. 435/69.4; 435/325; 435/320.1; 530/350; 536/23.5

(57) ABSTRACT

The present invention relates to a novel Cardiac And Pancreatic Protein (CAPP) which is a member of the muscle derived growth factor superfamily. In particular, isolated nucleic acid molecules are provided encoding the human CAPP protein. CAPP polypeptides are also provided as are vectors, host cells and recombinant methods for producing the same. The invention further relates to screening methods for identifying agonists and antagonists of CAPP activity. Also provided are diagnostic methods for detecting the presence of activated T-cells, and mature heart, pancreas and placental tissues and cells. The CAPP polypeptides can also be employed in cell culture media for regulating cell differentiation and maintaining heart, placenta and pancreas cells.

The present application claims the benefit of the filing date of provisional application 60/042,855 filed Mar. 28, 1997, which is herein incorporated by reference in its entirety.

### BACKGROUND OF THE INVENTION

1. Field of the Invention

The present invention relates to a novel muscle derived growth factor. More specifically, isolated nucleic acid molecules are provided encoding human Cardiac And Pancreatic Protein (CAPP). CAPP polypeptides are also provided, as are vectors, host cells and recombinant methods for producing the same. Also provided are diagnostic methods for detecting CAPP gene expression and methods for stimulating and inhibiting the growth of certain cells.

2. Related Art

Control of cell division is a basic aspect of multicellular existence that depends upon a programmed series of events. One factor in cellular proliferation and its control that has been identified is the presence of various polypeptide growth factors. Growth factors are essential components of growth media for in vitro cell culture and are involved in cell survival in vivo. Some of the growth factors that have been identified to date include PDGF (platelet-derived growth factor) implicated in the repair of the vascular system in vivo; EGF (epidermal growth factor) which acts as a mitogen for cells of ectodermal and mesodermal origin; TGF-α (transforming growth factor) which acts as a mitogen similarly to EGF but can make normal cells grow in agar; TGF-62 (transforming growth factor) which is a mitogen for some cells and a growth inhibitor for others; and NGF (nerve growth factor) involved in the development and maintenance of sympathetic and embryonic neurons. Watson et al., Molecular Biology of the Gene, p. 975 (Benjamin/Cummings 1987).

It is clear that particular cell types require particular growth factors. Peptide growth factors are produced and secreted from a variety of tissues. The target cells are typically located close to the site of release of the growth factor (paracrine response). In addition to their growth promoting and differentiation-inducing activities, growth factors elicit a wide variety of effects in their target cells and are involved in processes such as inflammation, immune reactions and wound repair. See, Pimentel, E. Handbook of Growth Factors, Volume 1: General Basics (CRC Press 1994).

Myocardial hypertrophy refers to a focal or general enlargement of the heart. Normal hypertrophy is a compensatory function to maintain the pumping function of the heart. Abnormal hypertrophy occurs in hypertension, myocardial infarction, valve disease and cardiomyopathy. Simpson, P. C. Heart Failure 5:113 (1989). Cardiac myocytes have been shown to be targets for the effects of peptide growth factors on differentiated gene expression. Stimulation of the α₁-adrenergic receptor induces hypertrophy of cultured cardiac myocytes and produces specific changes in gene expression at the level of transcription. Simpson, P. C.“Cardiac Myocyte Hypertrophy,” Molecular Biology of the Cardiovascular System, Roberts, R. et al. ed.: 125-133 (1990). In cardiac myocytes, the growth factors TGF-β1 and basic FGF concomitantly elicit complex and heterogeneous responses: selective inhibition of certain adult transcripts, concurrent with the up-regulation of “fetal” contractile protein genes. Schneider et al., “Oncogenes and Myogenesis,” Molecular Biology of the Cardiovascular System, Roberts, R. et al. ed.:63-71 (1990).

Monitoring of growth factor gene expression in myocytes and the other cells of the heart, including connective tissue, would be useful in detecting and studying abnormal hypertrophy both in vitro and in vivo. Organ and clonal cell systems have been developed to analyze cardiomyogenic differentiation. See, for example, Bader, D. et al., Molecular Biology of the Cardiovascular System, Roberts, R. et al. ed.:4149 (1990). Differentiation in these systems can be monitored by in vitro analysis of cardiac myogenesis and monoclonal antibodies that have been raised against muscle-specific proteins.

Additionally, polypeptide growth factors are very important cell culture reagents for stimulating cellular growth and aiding survival of the cells in vitro.

The search continues to exist for polypeptides that stimulate and/or inhibit growth of particular cells for both in vitro and in vivo uses. In addition, the search continues for novel tissue specific markers that can be employed qualitatively to help identify a particular cell or tissue type and employed qualitatively to assess whether cells, tissues or organs are abnormal in their expression of a particular polypeptide.

### SUMMARY OF THE INVENTION

The present invention provides isolated nucleic acid molecules comprising a polynucleotide encoding CAPP polypeptide having the amino acid sequence is shown in FIG. 1 (SEQ ID NO:2) or the amino acid sequence encoded by the cDNA clone deposited in a bacterial host as ATCC Deposit Number 97729 on Sep. 23, 1996. The nucleotide sequence determined by sequencing the deposited CAPP clone, which is shown in FIG. 1 (SEQ ID NO:1), contains an open reading frame encoding a polypeptide of 397 amino acid residues, including an initiation codon at positions 1-3, with a leader sequence of about 32 amino acid residues, and a predicted molecular weight of about 40 kDa. The amino acid sequence of the mature CAPP protein is shown in FIG. 1, amino acid residues 1-365 in SEQ ID NO:2.

Thus, one aspect of the invention provides an isolated nucleic acid molecule comprising a polynucleotide having a nucleotide sequence selected from the group consisting of: (a) a nucleotide sequence encoding the CAPP polypeptide having the complete amino acid sequence in FIG. 1 (SEQ ID NO:2); (b) a nucleotide sequence encoding the mature CAPP polypeptide having the amino acid sequence at positions 33-397 in FIG. 1 or 1-365 in SEQ ID NO:2; (c) a nucleotide sequence encoding the CAPP polypeptide having the complete amino acid sequence encoded by the cDNA clone contained in ATCC Deposit No. 97729; (d) a nucleotide sequence encoding the mature CAPP polypeptide having the amino acid sequence encoded by the cDNA clone contained in ATCC Deposit No. 97729; and (e) a nucleotide sequence complementary to any of the nucleotide sequences in (a), (b), (c) or (d) above.

Further embodiments of the invention include isolated nucleic acid molecules that comprise a polynucleotide having a nucleotide sequence at least 90% identical, and more preferably at least 95%, 96%, 97%, 98% or 99% identical, to any of the nucleotide sequences in (a), (b), (c), (d) or (e), above, or a polynucleotide which hybridizes under stringent hybridization conditions to a polynucleotide in (a), (b), (c), (d) or (e), above. This polynucleotide which hybridizes does not hybridize under stringent hybridization conditions to a polynucleotide having a nucleotide sequence consisting of only A residues or of only T residues. An additional nucleic acid embodiment of the invention relates to an isolated nucleic acid molecule comprising a polynucleotide which encodes the amino acid sequence of an epitope-bearing portion of a CAPP polypeptide having an amino acid sequence in (a), (b), (c) or (d), above.

The present invention also relates to recombinant vectors, which include the isolated nucleic acid molecules of the present invention, and to host cells containing the recombinant vectors, as well as to methods of making such vectors and host cells and for using them for production of CAPP polypeptides or peptides by recombinant techniques.

The invention further provides an isolated CAPP polypeptide having an amino acid sequence selected from the group consisting of: (a) the amino acid sequence of the CAPP polypeptide having the complete 397 amino acid sequence, including the leader sequence shown in FIG. 1 (SEQ ID NO:2); (b) the amino acid sequence of (b) the amino acid sequence of the mature CAPP polypeptide (without the leader) having the amino acid sequence at positions 1-365 in SEQ ID NO:2; (c) the amino acid sequence of the CAPP polypeptide having the complete amino acid sequence, including the leader, encoded by the cDNA clone contained in ATCC Deposit No. 97729; and (d) the amino acid sequence of the mature CAPP polypeptide having the amino acid sequence encoded by the cDNA clone contained in ATCC Deposit No. 97729. The polypeptides of the present invention also include polypeptides having an amino acid sequence with at least 90% similarity, and more preferably at least 95% similarity to those described in (a), (b), (c) or (d) above, as well as polypeptides having an amino acid sequence at least 80% identical, more preferably at least 90% identical, and still more preferably 95%, 96%, 97%, 98% or 99% identical to those above.

An additional embodiment of this aspect of the invention relates to a peptide or polypeptide which has the amino acid sequence of an epitope-bearing portion of a CAPP polypeptide having an amino acid sequence described in (a), (b), (c) or (d), above. Peptides or polypeptides having the amino acid sequence of an epitope-bearing portion of a CAPP polypeptide of the invention include portions of such polypeptides with at least six or seven, preferably at least nine, and more preferably at least about 30 amino acids to about 50 amino acids, although epitope-bearing polypeptides of any length up to and including the entire amino acid sequence of a polypeptide of the invention described above also are included in the invention. In another embodiment, the invention provides an isolated antibody that binds specifically to a CAPP polypeptide having an amino acid sequence described in (a), (b), (c) or (d) above.

The invention further provides methods for isolating antibodies that bind specifically to a CAPP polypeptide having an amino acid sequence as described herein. Such antibodies are useful diagnostically or therapeutically as describe below.

The expression of CAPP protein is expected to be necessary for the survival and maintenance of mature muscle cells, especially heart, placenta and pancreas tissue. Under certain conditions the CAPP protein is expected to act with other growth factors to modulate, e.g. block, the proliferation of mature heart and pancreas cells. Under certain conditions the CAPP protein is expected to act with other growth factors to program the differentiation of immature cells into cardiac or pancreatic cells. These functional properties of this peptide can be exploited in vivo in a number of useful ways. An antagonist of the CAPP protein may be useful in allowing mature muscle cells, such as myocytes to replicate and divide, something that does not occur in most normal myocytes. In vitro the CAPP protein can be employed to cause embryonic cells to differentiate into cardiac cells and to maintain cell cultures of cardiac, pancreatic or placental cells.

The CAPP gene was discovered in an activated T-cell cDNA library. CAPP gene expression and translation can be used as a marker to detect activated T-cells. Monitoring T cells activation is useful for a number of in vitro diagnostic purposes, including studying the effects of candidate drugs on the immune system, and determining whether the T cells of a subject have been activated by analyzing a blood sample taken from the subject or by assessing activity in an in vitro screening test.

The present inventors have discovered that CAPP is highly expressed in adult heart, pancreas and placenta tissue. For a number of disorders of smooth muscle tissue in the heart, pancreas or placenta, it is believed that significantly higher or lower levels of CAPP gene expression can be detected in certain tissues (e.g., heart, pancreas and placenta) or bodily fluids (e.g., serum, plasma, urine, synovial fluid or spinal fluid) taken from an individual having such a disorder, relative to a “standard” CAPP gene expression level, i.e., the CAPP expression level in tissue or bodily fluids from an individual not having a disorder of the heart, pancreas or placenta. Thus, the invention provides a diagnostic method useful during diagnosis of an internal organ disorder, wherein said disorder relates to the smooth muscle tissue of the heart, pancreas or placenta, which involves: (a) assaying CAPP gene expression level in cells or body fluid of an individual; (b) comparing the CAPP gene expression level with a standard CAPP gene expression level, whereby an increase or decrease in the assayed CAPP gene expression level compared to the standard expression level is indicative of one of said disorders.

Additionally, this CAPP gene expression can be employed as a marker to determine the presence of mature, terminally differentiated organ tissue, especially heart, pancreatic and placental tissue. Such a marker possesses practical utility in monitoring the growth of heart, pancreas and placental cells and tissues ex vivo. The effects of small molecule drugs and polypeptide growth factors on the development of these cells and tissues can be assessed by monitoring the level of expression of the CAPP gene.

The present invention also provides a screening method for identifying compounds capable of enhancing or inhibiting a cellular response induced by the CAPP polypeptide, which involves contacting cells which express the CAPP polypeptide with the candidate compound, assaying a cellular response, and comparing the cellular response to a standard cellular response, the standard being assayed when contact is made in absence of the candidate compound; whereby, an increased cellular response over the standard indicates that the compound is an agonist and a decreased cellular response over the standard indicates that the compound is an antagonist.

In another aspect, a screening assay for agonists and antagonists is provided which involves determining the effect a candidate compound has on CAPP polypeptide modulation of cellular growth and differentiation. In particular, the method involves contacting a cell culture with CAPP polypeptide and a candidate compound and determining whether CAPP polypeptide increases or decreases cellular differentiation or proliferation in the presence of the candidate compound.

An additional aspect of the invention is related to a method for treating an individual in need of an increased level of CAPP activity in the body comprising administering to such an individual a composition comprising a therapeutically effective amount of an isolated CAPP polypeptide of the invention or an agonist thereof.

A still further aspect of the invention is related to a method for treating an individual in need of a decreased level of CAPP activity in the body comprising, administering to such an individual a composition comprising a therapeutically effective amount of a CAPP antagonist. Preferred antagonists for use in the present invention are CAPP-specific antibodies.

### BRIEF DESCRIPTION OF THE FIGURES

FIG. 1 shows the nucleotide (SEQ ID NO:1) and deduced amino acid (SEQ ID NO:2) sequences of CAPP. The protein has a leader sequence of about 32 amino acid residues (underlined) and a deduced molecular weight of about 40 kDa. The predicted amino acid sequence of the mature CAPP protein is also shown in FIG. 1 (SEQ ID NO:2).

FIG. 2 shows the regions of similarity between the amino acid sequences of the CAPP protein and Drosophila “brainiac” gene (SEQ ID NO:3).

FIG. 3 shows an analysis of the CAPP amino acid sequence. Alpha, beta, turn and coil regions; hydrophilicity and hydrophobicity; amphipathic regions; flexible regions; antigenic index and surface probability are shown. In the “Antigenic Index—Jameson-Wolf” graph, the following amino acid residues in FIG. 1 correspond to the shown highly antigenic regions of the CAPP protein.

FIG. 4 shows a schematic representation of the pHE4a expression vector (SEQ ID NO:8). The locations of the kanamycin resistance marker gene, the multiple cloning site linker region, the oriC sequence, and the lacIq coding sequence are indicated.

FIG. 5 shows the nucleotide sequence of the regulatory elements of the pHE4a promoter (SEQ ID NO:9). The two lac operator sequences, the Shine-Delgarno sequence (S/D), and the terminal HindIII and NdeI restriction sites (italicized) are indicated.

### DETAILED DESCRIPTION

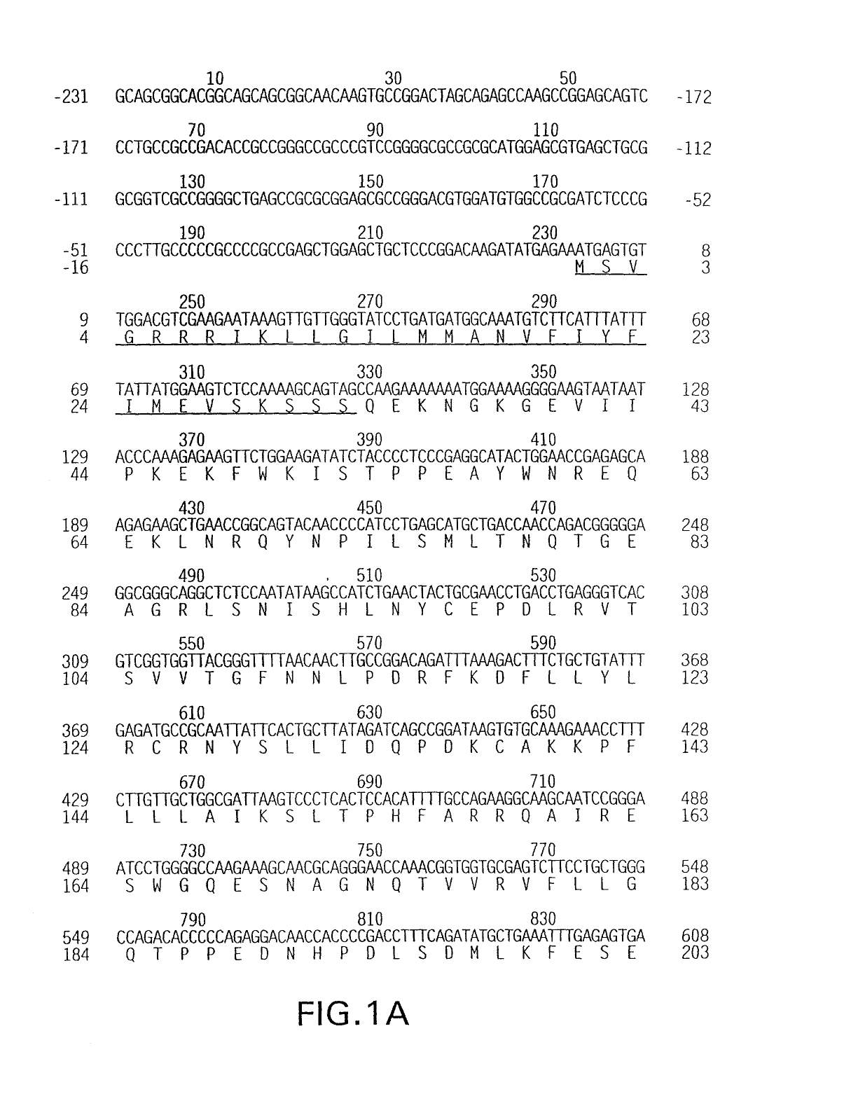

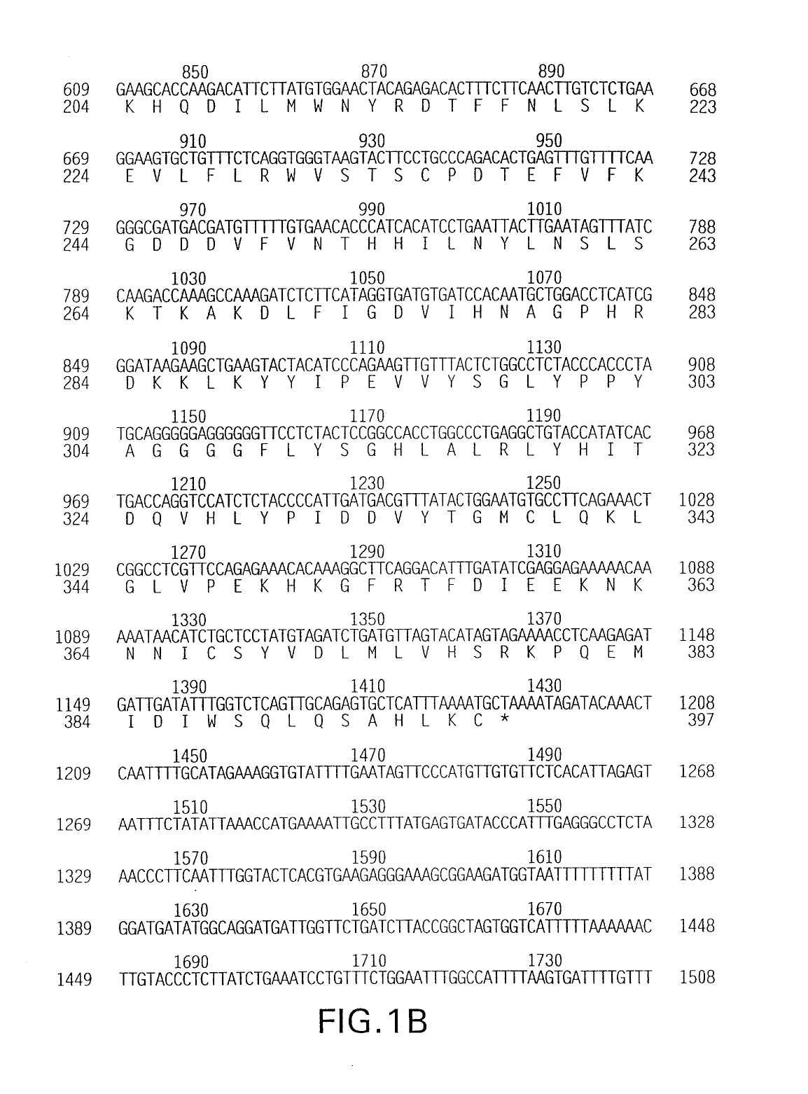

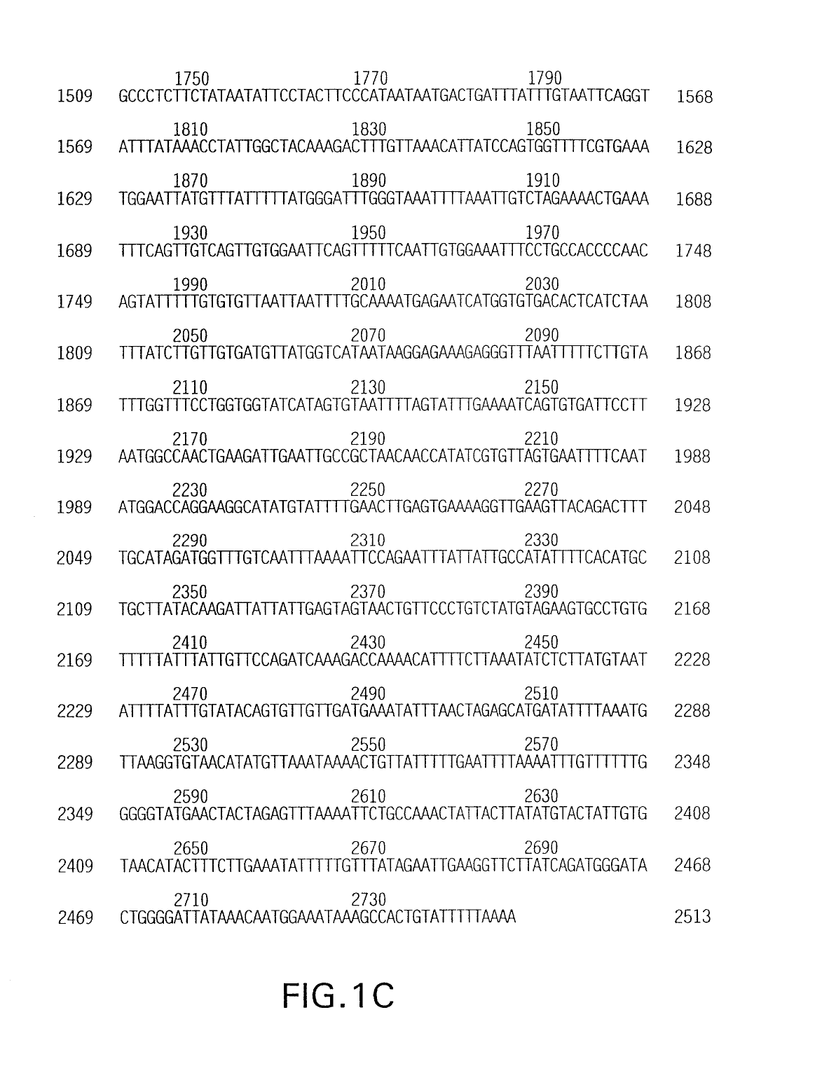

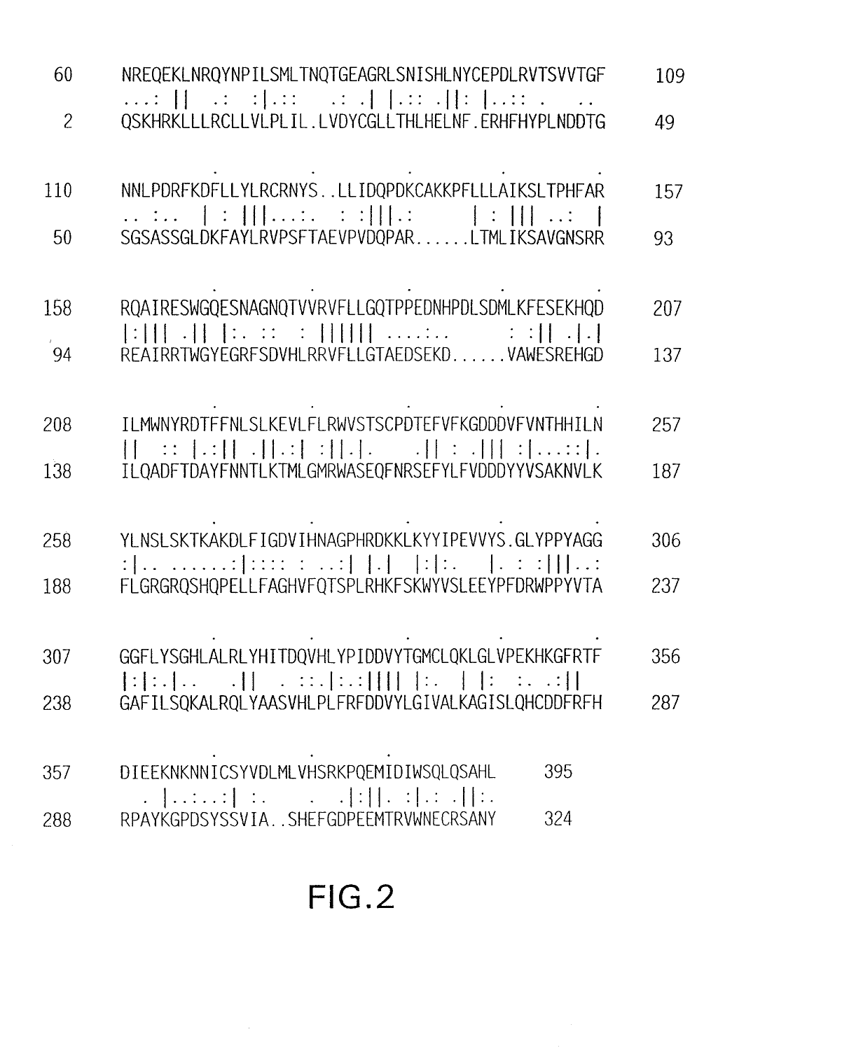

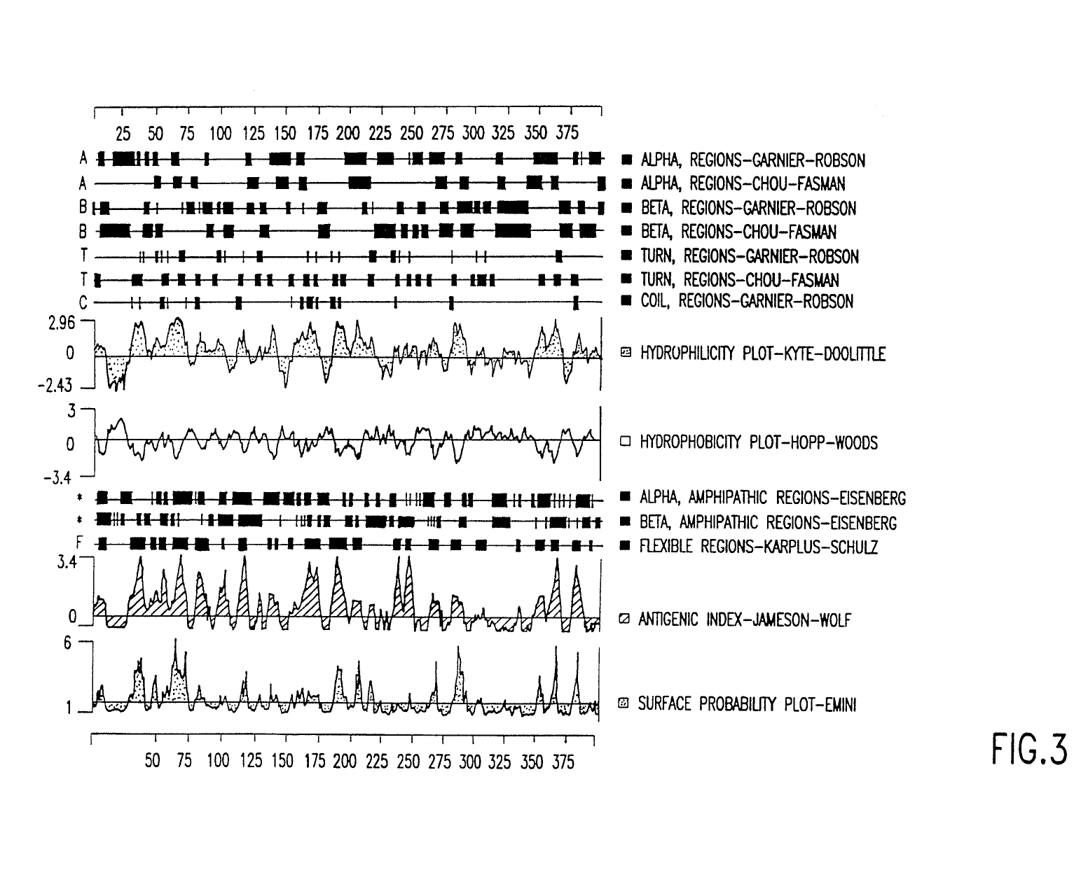

The present invention provides isolated nucleic acid molecules comprising a polynucleotide encoding a CAPP polypeptide having the amino acid sequence shown in FIG. 1 (SEQ ID NO:2), which was determined by sequencing a cloned cDNA. The CAPP protein of the present invention shares sequence homology with Drosophila “brainiac” gene (FIG. 2) (SEQ ID NO:3). The nucleotide sequence shown in FIG. 1 (SEQ ID NO:1) was obtained by sequencing the HTAAW41 clone, which was deposited on Sep. 23, 1996 at the American Type Culture Collection, 12301 Parklawn Drive, Rockville, Md. 20852, and given accession number 97729. The deposited clone is contained in the pBluescript SK(−) plasmid (Stratagene, La Jolla, Calif.).

### Nucleic Acid Molecules

Unless otherwise indicated, all nucleotide sequences determined by sequencing a DNA molecule herein were determined using an automated DNA sequencer (such as the Model 373 from Applied Biosystems, Inc.), and all amino acid sequences of polypeptides encoded by DNA molecules determined herein were predicted by translation of a DNA sequence determined as above. Therefore, as is known in the art for any DNA sequence determined by this automated approach, any nucleotide sequence determined herein may contain some errors. Nucleotide sequences determined by automation are typically at least about 90% identical, more typically at least about 95% to at least about 99.9% identical to the actual nucleotide sequence of the sequenced DNA molecule. The actual sequence can be more precisely determined by other approaches including manual DNA sequencing methods well known in the art. As is also known in the art, a single insertion or deletion in a determined nucleotide sequence compared to the actual sequence will cause a frame shift in translation of the nucleotide sequence such that the predicted amino acid sequence encoded by a determined nucleotide sequence will be completely different from the amino acid sequence actually encoded by the sequenced DNA molecule, beginning at the point of such an insertion or deletion.

Unless otherwise indicated, each “nucleotide sequence” set forth herein is presented as a sequence of deoxyribonucleotides (abbreviated A, G, C and T). However, by “nucleotide sequence” of a nucleic acid molecule or polynucleotide is intended, for a DNA molecule or polynucleotide, a sequence of deoxyribonucleotides, and for an RNA molecule or polynucleotide, the corresponding sequence of ribonucleotides (A, G, C and U), where each thymidine deoxyribonucleotide (1) in the specified deoxyribonucleotide sequence is replaced by the ribonucleotide uridine (U). For instance, reference to an RNA molecule having the sequence of SEQ ID NO:1 set forth using deoxyribonucleotide abbreviations is intended to indicate an RNA molecule having a sequence in which each deoxyribonucleotide A, G or C of SEQ ID NO:1 has been replaced by the corresponding ribonucleotide A, G or C, and each deoxyribonucleotide T has been replaced by a ribonucleotide U.

Using the information provided herein, such as the nucleotide sequence in FIG. 1, a nucleic acid molecule of the present invention encoding a CAPP polypeptide may be obtained using standard cloning and screening procedures, such as those for cloning cDNAs using mRNA as starting material. Illustrative of the invention, the nucleic acid molecule described in FIG. 1 (SEQ ID NO:1) was discovered in a cDNA library derived from activated T cells. By Northern blot analysis it has been determined that this gene is abundant in adult heart and pancreas, with low amounts in placenta, lung, liver, skeletal muscle, kidney, spleen, thymus, prostate, testis, ovary, small intestine, colon and peripheral blood leukocytes. The gene was identified by database distribution in activated T cells (3), CD34 positive cells, Ntera2 cells 14 days after RA stimulation, kidney cortex, adult heart, Jurkat cells and small intestine. The determined nucleotide sequence of the CAPP cDNA of FIG. 1 (SEQ ID NO:1) contains an open reading frame encoding a protein of 397 amino acid residues, with an initiation codon at positions 233-236 of the nucleotide sequence in FIG. 1 (SEQ ID NO:1), a predicted leader sequence of about 32 amino acid residues, and a deduced molecular weight of about 40 kDa. The amino acid sequence of the predicted mature CAPP is shown in 1 to residue 365 in SEQ ID NO:2. The CAPP protein shown in FIG. 1 (SEQ ID NO:2) is about 27.8% identical and about 48.4% similar to Drosophila “brainiac” protein (FIG. 2). This protein interacts with the EGF receptor pathway in follicle cell development (Goode et al, Development 116:177-192 (1992).

As one of ordinary skill would appreciate, due to the possibilities of sequencing errors discussed above, as well as the variability of cleavage sites for leaders in different known proteins, the actual CAPP polypeptide encoded by the deposited cDNA comprises about 397 amino acids, but may be anywhere in the range of 385410 amino acids; and the actual leader sequence of this protein is about 32 amino acids, but may be anywhere in the range of about 25 to about 40 amino acids.

As indicated, nucleic acid molecules of the present invention may be in the form of RNA, such as mRNA, or in the form of DNA, including, for instance, cDNA and genomic DNA obtained by cloning or produced synthetically. The DNA may be double-stranded or single-stranded. Single-stranded DNA or RNA may be the coding strand, also known as the sense strand, or it may be the non-coding strand, also referred to as the anti-sense strand.

By “isolated” nucleic acid molecule(s) is intended a nucleic acid molecule, DNA or RNA, which has been removed from its native environment For example, recombinant DNA molecules contained in a vector are considered isolated for the purposes of the present invention. Further examples of isolated DNA molecules include recombinant DNA molecules maintained in heterologous host cells or purified (partially or substantially) DNA molecules in solution. Isolated RNA molecules include in vivo or in vitro RNA transcripts of the DNA molecules of the present invention. Isolated nucleic acid molecules according to the present invention further include such molecules produced synthetically.

Isolated nucleic acid molecules of the present invention include DNA molecules comprising an open reading frame (ORF) with an initiation codon at positions 233-236 of the nucleotide sequence shown in FIG. 1 (SEQ ID NO:1); DNA molecules comprising the coding sequence for the mature CAPP protein shown in FIG. 1 (last 365 amino acids) (SEQ ID NO:2); and DNA molecules which comprise a sequence substantially different from those described above but which, due to the degeneracy of the genetic code, still encode the CAPP protein. Of course, the genetic code is well known in the art. Thus, it would be routine for one skilled in the art to generate the degenerate variants described above.

In addition, the invention present inventors have identified the following cDNA clones related to extensive portions of SEQ ID NO:1: HAHAA70F (SEQ ID NO:10), HTAAW41R (SEQ ID NO:11), HTABE60R (SEQ ID NO:12), HJUBA94R (SEQ ID NO:13), HSIBA68R (SEQ ID NO:14), and HSIBA68F (SEQ ID NO:15).

In addition, the invention present inventors have identified the following public cDNA clones related to extensive portions of SEQ ID NO:1: AA773646 (SEQ ID NO:16), AA449869 (SEQ ID NO:17), N66915 (SEQ ID NO:18), H93550 (SEQ ID NO:19), W26453 (SEQ ID NO:20), H13125 (SEQ ID NO:21), N51037 (SEQ ID NO:22), N58174 (SEQ ID NO:23), R74552 (SEQ ID NO:24), R82733 (SEQ ID NO:25), H78875 (SEQ ID NO:25), H47991 (SEQ ID NO:26), R74454 (SEQ ID NO:27), C20629 (SEQ ID NO:28), AA310578 (SEQ ID NO:29), AA263148 (SEQ ID NO:30), H00589 (SEQ ID NO:31), R31680 (SEQ ID NO:32), AA381631 (SEQ ID NO:33), H80116 (SEQ ID NO:34), H47990 (SEQ ID NO:35), AA381830 (SEQ ID NO:36), AA377082 (SEQ ID NO:37), H13126 (SEQ ID NO:38), R31722 (SEQ ID NO:39), AA377081 (SEQ ID NO:40), and D87736 (SEQ ID NO:41).

In another aspect, the invention provides isolated nucleic acid molecules encoding the CAPP polypeptide having an amino acid sequence encoded by the cDNA clone contained in the plasmid deposited as ATCC Deposit No. 97729 on Sep. 23,1996. Preferably, this nucleic acid molecule will encode the mature polypeptide encoded by the above-described deposited cDNA clone. The invention further provides an isolated nucleic acid molecule having the nucleotide sequence shown in FIG. 1 (SEQ ID NO:1) or the nucleotide sequence of the CAPP cDNA contained in the above-described deposited clone, or a nucleic acid molecule having a sequence complementary to one of the above sequences. Such isolated molecules, particularly DNA molecules, are useful as probes for gene mapping, by in situ hybridization with chromosomes, and for detecting expression of the CAPP gene in human tissue, for instance, by Northern blot analysis.

The present invention is further directed to fragments of the isolated nucleic acid molecules described herein. By a fragment of an isolated nucleic acid molecule having the nucleotide sequence of the deposited cDNA or the nucleotide sequence shown in FIG. 1 (SEQ ID NO:1) is intended fragments at least about 15 nt, and more preferably at least about 20 nt, still more preferably at least about 30 nt, and even more preferably, at least about 40 nt in length which are useful as diagnostic probes and primers as discussed herein. Of course, larger fragments 50, 75, 100, 125, 150, 175, 200, 225, 250, 275, 300, 325, 350, 375, 400, 425, 450, 475, 500, 525, 550, 575, 600, 625, 650, 675, 700, 725, 750, 775, 800, 825, 850, 875, 900, 925, 950, 975, 1000, 1025, 1050, 1075, 1100, 1125, 1150, 1175, 1200, 1225, 1250, 1275, 1300, 1325, 1350, 1375, 1400, 1425, 1450,1475, 1500, 1525, 1550, 1575, 1600, 1625, 1650, 1675, 1700, 1725, 1750, 1775, 1800, 1825, 1850, 1875, 1900, 1925, 1950, 1975, 2000, 2025, 2050, 2075, 2100, 2125, 2150, 2175, 2200, 2225, 2250, 2275, 2300, 2325, 2350, 2375, 2400, 2425, 2450, 2475, 2500, 2525, 2550, 2575, 2600, 2625, 2650, 2675, 2700, 2725 and 2740 nt in length are also useful according to the present invention as are fragments corresponding to most, if not all, of the nucleotide sequence of the deposited cDNA or as shown in FIG. 1 (SEQ ID NO:1). By a fragment at least 20 nt in length, for example, is intended fragments which include 20 or more contiguous bases from the nucleotide sequence of the deposited cDNA or the nucleotide sequence as shown in FIG. 1 (SEQ ID NO:1). Since the gene has been deposited and the nucleotide sequence shown in FIG. 1 (SEQ ID NO:1) is provided, generating such DNA fragments would be routine to the skilled artisan. For example, restriction endonuclease cleavage or shearing by sonication could easily be used to generate fragments of various sizes. Alternatively, such fragments could be generated synthetically.

Preferred nucleic acid fragments of the present invention include nucleic acid molecules encoding epitope-bearing portions of the CAPP protein. In particular, such nucleic acid fragments of the present invention include nucleic acid molecules encoding: a polypeptide comprising amino acid residues from about −32 to about −22 in SEQ ID NO:2; a polypeptide comprising amino acid residues from about −4 to about 40 in SEQ ID NO:2; a polypeptide comprising amino acid residues from about 46 to about 57 in SEQ ID NO:2; a polypeptide comprising amino acid residues from about 62 to about 73 in SEQ ID NO:2; a polypeptide comprising amino acid residues from about 78 to about 87 in SEQ ID NO:2; a polypeptide comprising amino acid residues from about 92 to about 110 in SEQ ID NO:2; a polypeptide comprising amino acid residues from about 119 to about 144 in SEQ ID NO:2; a polypeptide comprising amino acid residues from about 152 to about 186 in SEQ ID NO:2; a polypeptide comprising amino acid residues from about 200 to about 219 in SEQ ID NO:2; a polypeptide comprising amino acid residues from about 230 to about 240 in SEQ ID NO:2; a polypeptide comprising amino acid residues from about 248 to about 258 in SEQ ID NO:2; a polypeptide comprising amino acid residues from about 314 to about 336 in SEQ ID NO:2; and a polypeptide comprising amino acid residues from about 344 to about 353 in SEQ ID NO:2. The inventors have determined that the above polypeptide fragments are antigenic regions of the CAPP protein. Methods for determining other such epitope-bearing portions of the CAPP protein are described in detail below.

In another aspect, the invention provides an isolated nucleic acid molecule comprising a polynucleotide which hybridizes under stringent hybridization conditions to a portion of the polynucleotide in a nucleic acid molecule of the invention described above, for instance, the cDNA clone contained in ATCC Deposit 97729. By “stringent hybridization conditions” is intended overnight incubation at 42° C. in a solution comprising: 50% formamide, 5×SSC (150 mM NaCl, 15 mM trisodium citrate), 50 mM sodium phosphate (pH 7.6), 5×Denhardt's solution, 10% dextran sulfate, and 20 mg/ml denatured, sheared salmon sperm DNA, followed by washing the filters in 0.1×SSC at about 65° C.

By a polynucleotide which hybridizes to a “portion” of a polynucleotide is intended a polynucleotide (either DNA or RNA) hybridizing to at least about 15 nucleotides (nt), and more preferably at least about 20 nt, still more preferably at least about 30 nt, and even more preferably about 30-70 nt of the reference polynucleotide. These are useful as diagnostic probes and primers as discussed above and in more detail below.

Of course, polynucleotides hybridizing to a larger portion of the reference polynucleotide (e.g., the deposited cDNA clone), for instance, a portion 50-750 nt in length, or even to the entire length of the reference polynucleotide, are also useful as probes according to the present invention, as are polynucleotides corresponding to most, if not all, of the nucleotide sequence of the deposited cDNA or the nucleotide sequence as shown in FIG. 1 (SEQ ID NO:1). By a portion of a polynucleotide of “at least 20 nt in length,” for example, is intended 20 or more contiguous nucleotides from the nucleotide sequence of the reference polynucleotide (e.g., the deposited cDNA or the nucleotide sequence as shown in FIG. 1 (SEQ ID NO:1)). As indicated, such portions are useful diagnostically either as a probe according to conventional DNA hybridization techniques or as primers for amplification of a target sequence by the polymerase chain reaction (PCR), as described, for instance, in Molecular Cloning, A Laboratory Manual, 2nd. edition, edited by Sambrook, J., Fritsch, E. F. and Maniatis, T., (1989), Cold Spring Harbor Laboratory Press, the entire disclosure of which is hereby incorporated herein by reference.

Since a CAPP cDNA clone has been deposited and its determined nucleotide sequence is provided in FIG. 1 (SEQ ID NO:1), generating polynucleotides which hybridize to a portion of the CAPP cDNA molecule would be routine to the skilled artisan. For example, restriction endonuclease cleavage or shearing by sonication of the CAPP cDNA clone could easily be used to generate DNA portions of various sizes which are polynucleotides that hybridize to a portion of the CAPP cDNA molecule. Alternatively, the hybridizing polynucleotides of the present invention could be generated synthetically according to known techniques. Of course, a polynucleotide which hybridizes only to a poly A sequence (such as the 3′ terminal poly(A) tract of the CAPP cDNA shown in FIG. 1 (SEQ ID NO:1)), or to a complementary stretch of T (or U) resides, would not be included in a polynucleotide of the invention used to hybridize to a portion of a nucleic acid of the invention, since such a polynucleotide would hybridize to any nucleic acid molecule containing a poly (A) stretch or the complement thereof (e.g., practically any double-stranded cDNA clone).

As indicated, nucleic acid molecules of the present invention which encode a CAPP polypeptide may include, but are not limited to those encoding the amino acid sequence of the mature polypeptide, by itself; the coding sequence for the mature polypeptide and additional sequences, such as those encoding the about 32 amino acid leader or secretory sequence, such as a pre-, or pro- or prepro-protein sequence; the coding sequence of the mature polypeptide, with or without the aforementioned additional coding sequences, together with additional, non-coding sequences, including for example, but not limited to introns and non-coding 5 ′ and 3 ′ sequences, such as the transcribed, non-translated sequences that play a role in transcription, mRNA processing, including splicing and polyadenylation signals, for example—ribosome binding and stability of mRNA; an additional coding sequence which codes for additional amino acids, such as those which provide additional functionalities. Thus, the sequence encoding the polypeptide may be fused to a marker sequence, such as a sequence encoding a peptide which facilitates purification of the fused polypeptide. In certain preferred embodiments of this aspect of the invention, the marker amino acid sequence is a hexa-histidine peptide, such as the tag provided in a pQE vector (Qiagen, Inc.), among others, many of which are commercially available. As described in Gentz et al, Proc. Natl. Acad. Sci. USA 86:821-824 (1989), for instance, hexa-histidine provides for convenient purification of the fusion protein. The “HA” tag is another peptide useful for purification which corresponds to an epitope derived from the influenza hemagglutinin protein, which has been described by Wilson et al, Cell 37: 767 (1984). As discussed below, other such fusion proteins include the CAPP fused to Fc at the N- or C-terminus.

The present invention further relates to variants of the nucleic acid molecules of the present invention, which encode portions, analogs or derivatives of the CAPP protein. Variants may occur naturally, such as a natural allelic variant. By an “allelic variant” is intended one of several alternate forms of a gene occupying a given locus on a chromosome of an organism. Genes H, Lewin, B., ed., John Wiley & Sons, New York (1985). Non-naturally occurring variants may be produced using art-known mutagenesis techniques.

Such variants include those produced by nucleotide substitutions, deletions or additions. The substitutions, deletions or additions may involve one or more nucleotides. The variants may be altered in coding regions, non-coding regions, or both. Alterations in the coding regions may produce conservative or non-conservative amino acid substitutions, deletions or additions. Especially preferred among these are silent substitutions, additions and deletions, which do not alter the properties and activities of the CAPP protein or portions thereof. Also especially preferred in this regard are conservative substitutions. Most highly preferred are nucleic acid molecules encoding the mature protein having the amino acid sequence shown in FIG. 1 (SEQ ID NO:2) or the mature CAPP amino acid sequence encoded by the deposited cDNA clone.

Further embodiments of the invention include isolated nucleic acid molecules comprising a polynucleotide having a nucleotide sequence at least 90% identical, and more preferably at least 95%, 96%, 97%, 98% or 99% identical to (a) a nucleotide sequence encoding the polypeptide having the amino acid sequence in SEQ ID NO:2; (b) a nucleotide sequence encoding the polypeptide having the amino acid sequence in SEQ ID NO:2, but lacking the N-terminal methionine; (c) a nucleotide sequence encoding the polypeptide having the amino acid sequence at positions from about 1 to about 365 in SEQ ID NO:2; (d) a nucleotide sequence encoding the polypeptide having the amino acid sequence encoded by the cDNA clone contained in ATCC Deposit No. 97729; (e) a nucleotide sequence encoding the mature CAPP polypeptide having the amino acid sequence encoded by the cDNA clone contained in ATCC Deposit No. 97729; or (f) a nucleotide sequence complementary to any of the nucleotide sequences in (a), (b), (c), (d), or (e).

By a polynucleotide having a nucleotide sequence at least, for example, 95% “identical” to a reference nucleotide sequence encoding a CAPP polypeptide is intended that the nucleotide sequence of the polynucleotide is identical to the reference sequence except that the polynucleotide sequence may include up to five point mutations per each 100 nucleotides of the reference nucleotide sequence encoding the CAPP polypeptide. In other words, to obtain a polynucleotide having a nucleotide sequence at least 95% identical to a reference nucleotide sequence, up to 5% of the nucleotides in the reference sequence may be deleted or substituted with another nucleotide, or a number of nucleotides up to 5% of the total nucleotides in the reference sequence may be inserted into the reference sequence. These mutations of the reference sequence may occur at the 5′ or 3′ terminal positions of the reference nucleotide sequence or anywhere between those terminal positions, interspersed either individually among nucleotides in the reference sequence or in one or more contiguous groups within the reference sequence.

As a practical matter, whether any particular nucleic acid molecule is at least 90%, 95%, 96%, 97%, 98% or 99% identical to, for instance, the nucleotide sequence shown in FIG. 1 or to the nucleotides sequence of the deposited cDNA clone can be determined conventionally using known computer programs such as the Bestfit program (Wisconsin Sequence Analysis Package, Version 8 for Unix, Genetics Computer Group, University Research Park, 575 Science Drive, Madison, Wis. 53711. Bestfit uses the local homology algorithm of Smith and Waterman, Advances in Applied Mathematics 2:482-489(1981), to find the best segment of homology between two sequences. When using Bestfit or any other sequence alignment program to determine whether a particular sequence is, for instance, 95% identical to a reference sequence according to the present invention, the parameters are set, of course, such that the percentage of identity is calculated over the full length of the reference nucleotide sequence and that gaps in homology of up to 5% of the total number of nucleotides in the reference sequence are allowed.

The present application is directed to nucleic acid molecules at least 90%, 95%, 96%, 97%, 98% or 99% identical to the nucleic acid sequence shown in FIG. 1 (SEQ ID NO:1) or to the nucleic acid sequence of the deposited cDNA, irrespective of whether they encode a polypeptide having CAPP activity. This is because even where a particular nucleic acid molecule does not encode a polypeptide having CAPP activity, one of skill in the art would still know how to use the nucleic acid molecule, for instance, as a hybridization probe or a polymerase chain reaction (PCR) primer. Uses of the nucleic acid molecules of the present invention that do not encode a polypeptide having CAPP activity include, inter alia, (1) isolating the CAPP gene or allelic variants thereof in a cDNA library; (2) in situ hybridization (e.g., “FISH”) to metaphase chromosomal spreads to provide precise chromosomal location of the CAPP gene, as described in Verma et al., Human Chromosomes: A Manual of Basic Techniques, Pergamon Press, New York (1988); and Northern Blot analysis for detecting CAPP mRNA expression in specific tissues.

Preferred, however, are nucleic acid molecules having sequences at least 90%, 95%, 96%, 97%, 98% or 99% identical to the nucleic acid sequence shown in FIG. 1 (SEQ ID NO:1) or to the nucleic acid sequence of the deposited cDNA which do, in fact, encode a polypeptide having CAPP protein activity. By “a polypeptide having CAPP activity” is intended polypeptides exhibiting activity similar, but not necessarily identical, to an activity of the CAPP protein of the invention (either the full-length protein or, preferably, the mature protein), as measured in a particular biological assay.

Thus, “a polypeptide having CAPP protein activity” includes polypeptides that exhibit CAPP activity. Although the degree of activity need not be identical to that of the CAPP protein, preferably, “a polypeptide having CAPP protein activity” will exhibit substantially similar activity as compared to the CAPP protein (i.e., the candidate polypeptide will exhibit greater activity or not more than about twenty-fold less and, preferably, not more than about ten-fold less activity relative to the reference CAPP protein).

Of course, due to the degeneracy of the genetic code, one of ordinary skill in the art will immediately recognize that a large number of the nucleic acid molecules having a sequence at least 90%, 95%, 96%, 97%, 98% or 99% identical to the nucleic acid sequence of the deposited cDNA or the nucleic acid sequence shown in FIG. 1 (SEQ ID NO:1) will encode a polypeptide “having CAPP protein activity.” In fact, since degenerate variants of these nucleotide sequences all encode the same polypeptide, this will be clear to the skilled artisan even without performing the above described comparison assay. It will be further recognized in the art that, for such nucleic acid molecules that are not degenerate variants, a reasonable number will also encode a polypeptide having CAPP protein activity. This is because the skilled artisan is fully aware of amino acid substitutions that are either less likely or not likely to significantly effect protein function (e.g., replacing one aliphatic amino acid with a second aliphatic amino acid).

For example, guidance concerning how to make phenotypically silent amino acid substitutions is provided in Bowie, J. U. et al., “Deciphering the Message in Protein Sequences: Tolerance to Amino Acid Substitutions,” Science 247:1306-1310 (1990), wherein the authors indicate that there are two main approaches for studying the tolerance of an amino acid sequence to change. The first method relies on the process of evolution, in which mutations are either accepted or rejected by natural selection. The second approach uses genetic engineering to introduce amino acid changes at specific positions of a cloned gene and selections or screens to identify sequences that maintain functionality. As the authors state, these studies have revealed that proteins are surprisingly tolerant of amino acid substitutions. The authors further indicate which amino acid changes are likely to be permissive at a certain position of the protein. For example, most buried amino acid residues require nonpolar side chains, whereas few features of surface side chains are generally conserved. Other such phenotypically silent substitutions are described in Bowie, J. U. et al., supra, and the references cited therein.

### Vectors and Host Cells

The present invention also relates to vectors which include the isolated DNA molecules of the present invention, host cells which are genetically engineered with the recombinant vectors, and the production of CAPP polypeptides or fragments thereof by recombinant techniques.

Recombinant constructs may be introduced into host cells using well known techniques such infection, transduction, transfection, transvection, electroporation and transformation. The vector may be, for example, a phage, plasmid, viral or retroviral vector. Retroviral vectors may be replication competent or replication defective. In the latter case, viral propagation generally will occur only in complementing host cells.

The polynucleotides may be joined to a vector containing a selectable marker for propagation in a host. Generally, a plasmid vector is introduced in a precipitate, such as a calcium phosphate precipitate, or in a complex with a charged lipid. If the vector is a virus, it may be packaged in vitro using an appropriate packaging cell line and then transduced into host cells.

Preferred are vectors comprising cis-acting control regions to the polynucleotide of interest. Appropriate trans-acting factors may be supplied by the host, supplied by a complementing vector or supplied by the vector itself upon introduction into the host.

In certain preferred embodiments in this regard, the vectors provide for specific expression, which may be inducible and/or cell type-specific. Particularly preferred among such vectors are those inducible by environmental factors that are easy to manipulate, such as temperature and nutrient additives.

Expression vectors useful in the present invention include chromosomal-, episomal- and virus-derived vectors, e.g., vectors derived from bacterial plasmids, bacteriophage, yeast episomes, yeast chromosomal elements, viruses such as baculoviruses, papova viruses, vaccinia viruses, adenoviruses, fowl pox viruses, pseudorabies viruses and retroviruses, and vectors derived from combinations thereof, such as cosmids and phagemids.

The DNA insert should be operatively linked to an appropriate promoter, such as the phage lambda PL promoter, the E. coli lac, trp and tac promoters, the SV40 early and late promoters and promoters of retroviral LTRs, to name a few. Other suitable promoters will be known to the skilled artisan. The expression constructs will further contain sites for transcription initiation, termination and, in the transcribed region, a ribosome binding site for translation. The coding portion of the mature transcripts expressed by the constructs will preferably include a translation initiating at the beginning and a termination codon (UAA, UGA or UAG) appropriately positioned at the end of the polypeptide to be translated.

As indicated, the expression vectors will preferably include at least one selectable marker. Such markers include dihydrofolate reductase or neomycin resistance for eukaryotic cell culture and tetracycline or ampicillin resistance genes for culturing in E. coli and other bacteria. Representative examples of appropriate hosts include, but are not limited to, bacterial cells, such as E. coli, Streptomyces and Salmonella typhimurium cells; fungal cells, such as yeast cells; insect cells such as Drosophila S2 and Spodoptera Sf9 cells; animal cells such as CHO, COS and Bowes melanoma cells; and plant cells. Appropriate culture mediums and conditions for the above-described host cells are known in the art.

In addition to the use of expression vectors in the practice of the present invention, the present invention further includes novel expression vectors comprising operator and promoter elements operatively linked to nucleotide sequences encoding a protein of interest. One example of such a vector is pHE4a which is described in detail below.

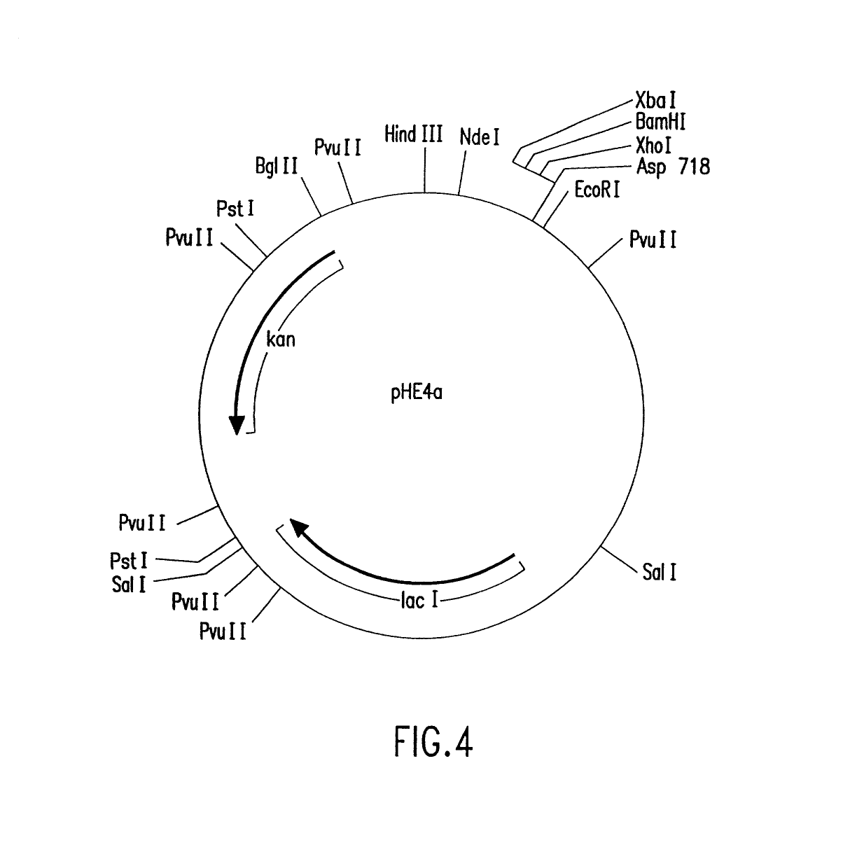

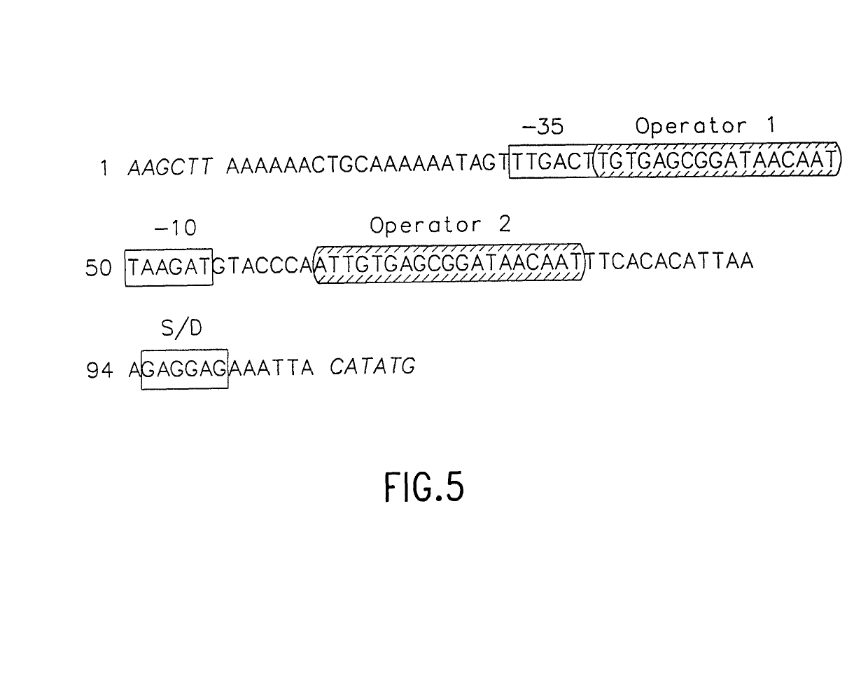

As summarized in FIGS. 4 and 5, components of the pHE4a vector (SEQ ID NO:8) include: 1) a neomycinphosphotransferase gene as a selection marker, 2) an E. coli origin of replication; 3) a T5 phage promoter sequence, 4) two lac operator sequences, 5) a Shine-Delgarno sequence, 6) the lactose operon repressor gene (lacIq) and 7) a multiple cloning site linker region. The origin of replication (oriC) is derived from pUC19 (LTI, Gaithersburg, Md.). The promoter sequence and operator sequences were made synthetically. Synthetic production of nucleic acid sequences is well known in the art. CLONTECH 95/96 Catalog, pages 215-216, CLONTECH, 1020 East Meadow Circle, Palo Alto, Calif. 94303. The pHE4a vector was deposited with the ATCC on Feb. 25, 1998, and given accession number 209645.

A nucleotide sequence encoding CAPP (SEQ ID NO:1), is operatively linked to the promoter and operator of pHE4a by restricting the vector with NdeI and either XbaI, BamHI, XhoI, or Asp718, and isolating the larger fiagment (the multiple cloning site region is about 310 nucleotides) on a gel. The nucleotide sequence encoding CAPP (SEQ ID NO:1) having the appropriate restriction sites is generated, for example, according to the PCR protocol described in Example 1, using PCR primers having restriction sites for NdeI (as the 5′ primer) and either XbaI, BamHI, XhoI, or Asp718 (as the 3′ primer). The PCR insert is gel purified and restricted with compatible enzymes. The insert and vector are ligated according to standard protocols.

As noted above, the pHE4a vector contains a lacIq gene. LacIq is an allele of the lacI gene which confers tight regulation of the lac operator. Amann, E. et al., Gene 69:301-315 (1988); Stark, M., Gene 51:255-267 (1987). The lacIq gene encodes a repressor protein which binds to lac operator sequences and blocks transcription of down-stream (i.e., 3′) sequences. However, the lacIq gene product dissociates from the lac operator in the presence of either lactose or certain lactose analogs, e.g., isopropyl B-D-thiogalactopyranoside (IPTG). CAPP thus is not produced in appreciable quantities in uninduced host cells containing the pHE4a vector. Induction of these host cells by the addition of an agent such as IPTG, however, results in the expression of the CAPP coding sequence.

The promoter/operator sequences of the pHE4a vector (SEQ ID NO:9) comprise a T5 phage promoter and two lac operator sequences. One operator is located 5′ to the transcriptional start site and the other is located 3′ to the same site. These operators, when present in combination with the lacIq gene product, confer tight repression of down-stream sequences in the absence of a lac operon inducer, e.g., IPTG. Expression of operatively linked sequences located down-stream from the lac operators may be induced by the addition of a lac operon inducer, such as IPTG. Binding of a lac inducer to the lacIq proteins results in their release from the lac operator sequences and the initiation of transcription of operatively linked sequences. Lac operon regulation of gene expression is reviewed in Devlin, T., TEXTBOOK OF BIOCHEMISTRY WITH CLINICAL CORRELATIONS, 4th Edition (1997), pages 802-807.

The pHE4 series of vectors contain all of the components of the pHE4a vector except for the CAPP coding sequence. Features of the pHE4a vectors include optimized synthetic T5 phage promoter, lac operator, and Shine-Delagarno sequences. Further, these sequences are also optimally spaced so that expression of an inserted gene may be tightly regulated and high level of expression occurs upon induction.

Among known bacterial promoters suitable for use in the production of proteins of the present invention include the E. coli lacI and lacZ promoters, the T3 and T7 promoters, the gpt promoter, the lambda PR and PL promoters and the trp promoter. Suitable eukaryotic promoters include the CMV immediate early promoter, the HSV thymidine kinase promoter, the early and late SV40 promoters, the promoters of retroviral LTRs, such as those of the Rous Sarcoma Virus (RSV), and metallothionein promoters, such as the mouse metallothionein-I promoter.

The pHE4a vector also contains a Shine-Delgarno sequence 5′ to the AUG initiation codon. Shine-Delgarno sequences are short sequences generally located about 10 nucleotides up-stream (i.e., 5′) from the AUG initiation codon. These sequences essentially direct prokaryotic ribosomes to the AUG initiation codon.

Thus, the present invention is also directed to expression vector useful for the production of the proteins of the present invention. This aspect of the invention is exemplified by the pHE4a vector (SEQ ID NO:8).

Among vectors preferred for use in bacteria include pQE70, pQE60 and pQE-9, available from Qiagen; pBS vectors, Phagescript vectors, Bluescript vectors, pNH8A, pNH16a, pNH18A, pNH46A, available from Stratagene; and ptrc99a, pKK223-3, pKK233-3, pDR540, pRIT5 available from Pharmacia. Among preferred eukaryotic vectors are pWLNEO, pSV2CAT, pOG44, pXT1 and pSG available from Stratagene; and pSVK3, pBPV, pMSG and pSVL available from Pharmacia. Other suitable vectors will be readily apparent to the skilled artisan.

Among known bacterial promoters suitable for use in the present invention include the E. coli lacI and lacZ promoters, the T3 and T7 promoters, the gpt promoter, the lambda PR and PL promoters and the trp promoter. Suitable eukaryotic promoters include the CMV immediate early promoter, the HSV thymidine kinase promoter, the early and late SV40 promoters, the promoters of retroviral LTRs, such as those of the Rous sarcoma virus (RSV), and metallothionein promoters, such as the mouse metallothionein-I promoter.

Introduction of the construct into the host cell can be effected by calcium phosphate transfection, DEAE-dextran mediated transfection, cationic lipid-mediated transfection, electroporation, transduction, infection or other methods. Such methods are described in many standard laboratory manuals, such as Davis et al., Basic Methods In Molecular Biology (1986).

Transcription of the DNA encoding the polypeptides of the present invention by higher eukaryotes may be increased by inserting an enhancer sequence into the vector. Enhancers are cis-acting elements of DNA, usually about from 10 to 300 bp that act to increase transcriptional activity of a promoter in a given host cell-type. Examples of enhancers include the SV40 enhancer, which is located on the late side of the replication origin at bp 100 to 270, the cytomegalovirus early promoter enhancer, the polyoma enhancer on the late side of the replication origin, and adenovirus enhancers.

For secretion of the translated protein into the lumen of the endoplasmic reticulum, into the periplasmic space or into the extracellular environment, appropriate secretion signals may be incorporated into the expressed polypeptide. The signals may be endogenous to the polypeptide or they may be heterologous signals.

The polypeptide may be expressed in a modified form, such as a fusion protein, and may include not only secretion signals, but also additional heterologous functional regions. For instance, a region of additional amino acids, particularly charged amino acids, may be added to the N-terminus of the polypeptide to improve stability and persistence in the host cell, during purification, or during subsequent handling and storage. Also, peptide moieties may be added to the polypeptide to facilitate purification. Such regions may be removed prior to final preparation of the polypeptide. The addition of peptide moieties to polypeptides to engender secretion or excretion, to improve stability and to facilitate purification, among others, are familiar and routine techniques in the art. A preferred fusion protein comprises a heterologous region from immunoglobulin that is useful to solubilize proteins. For example, EP A 0,464,533 (Canadian counterpart 2,045,869) discloses fusion proteins comprising various portions of constant region of immunoglobin molecules together with another human protein or part thereof. In many cases, the Fc part in a fusion protein is thoroughly advantageous for use in therapy and diagnosis and thus results, for example, in improved pharmacokinetic properties (EP A 0,232,262). On the other hand, for some uses it would be desirable to be able to delete the Fc part after the fusion protein has been expressed, detected and purified in the advantageous manner described. This is the case when Fc portion proves to be a hindrance to use in therapy and diagnosis, for example when the fusion protein is to be used as antigen for immunizations. In drug discovery, for example, human proteins, such as, hIL5-receptor has been fused with Fc portions for the purpose of high-throughput screening assays to identify antagonists of hIL-5. See, Bennett et al., Journal of Molecular Recognition 8:52-58 (1995) and Johanson et al., J. Biol. Chem. 270(16):9459-9471 (1995).

The CAPP protein can be recovered and purified from recombinant cell cultures by well-known methods including ammonium sulfate or ethanol precipitation, acid extraction, anion or cation exchange chromatography, phosphocellulose chromatography, hydrophobic interaction chromatography, affinity chromatography, hydroxylapatite chromatography and lectin chromatography. Most preferably, high performance liquid chromatography (“HPLC”) is employed for purification. Polypeptides of the present invention include naturally purified products, products of chemical synthetic procedures, and products produced by recombinant techniques from a prokaryotic or eukaryotic host, including, for example, bacterial, yeast, higher plant, insect and mammalian cells. Depending upon the host employed in a recombinant production procedure, the polypeptides of the present invention may be glycosylated or may be non-glycosylated. In addition, polypeptides of the invention may also include an initial modified methionine residue, in some cases as a result of host-mediated processes.

### CAPP Polypeptides and Fragments

The invention further provides an isolated CAPP polypeptide having the amino acid sequence encoded by the deposited cDNA, or the amino acid sequence in FIG. 1 (SEQ ID NO.2), or a peptide or polypeptide comprising a portion of the above polypeptides. The terms “peptide” and “oligopeptide” are considered synonymous (as is commonly recognized) and each term can be used interchangeably as the context requires to indicate a chain of at least to amino acids coupled by peptidyl linkages. The word “polypeptide” is used herein for chains containing more than ten amino acid residues. All oligopeptide and polypeptide formulas or sequences herein are written from left to right and in the direction from amino terminus to carboxy terminus.

It will be recognized in the art that some amino acid sequences of the CAPP polypeptide can be varied without significant effect of the structure or function of the protein. If such differences in sequence are contemplated, it should be remembered that there will be critical areas on the protein which determine activity. In general, it is possible to replace residues which form the tertiary structure, provided that residues performing a similar function are used. In other instances, the type of residue may be completely unimportant if the alteration occurs at a non-critical region of the protein.

Thus, the invention further includes variations of the CAPP polypeptide which show substantial CAPP polypeptide activity or which include regions of CAPP protein such as the protein portions discussed below. Such mutants include deletions, insertions, inversions, repeats, and type substitutions. As indicated above, guidance concerning which amino acid changes are likely to be phenotypically silent can be found in Bowie, J. U., et al, “Deciphering the Message in Protein Sequences: Tolerance to Amino Acid Substitutions,” Science 247:1306-1310 (1990).

Thus, the fragment, derivative or analog of the polypeptide of SEQ ID NO:2, or that encoded by the deposited cDNA, may be (i) one in which one or more of the amino acid residues are substituted with a conserved or non-conserved amino acid residue (preferably a conserved amino acid residue) and such substituted amino acid residue may or may not be one encoded by the genetic code, or (ii) one in which one or more of the amino acid residues includes a substituent group, or (iii) one in which the mature polypeptide is fused with another compound, such as a compound to increase the half-life of the polypeptide (for example, polyethylene glycol), or (iv) one in which the additional amino acids are fused to the mature polypeptide, such as an IgG Fc fusion region peptide or leader or secretory sequence or a sequence which is employed for purification of the mature polypeptide or a proprotein sequence. Such fragments, derivatives and analogs are deemed to be within the scope of those skilled in the art from the teachings herein.

Of particular interest are substitutions of charged amino acids with another charged amino acid and with neutral or negatively charged amino acids. The latter results in proteins with reduced positive charge to improve the characteristics of the CAPP protein. The prevention of aggregation is highly desirable. Aggregation of proteins not only results in a loss of activity but can also be problematic when preparing pharmaceutical formulations, because they can be immunogenic. (Pinckard et al., Clin. Exp. Immunol. 2:331-340 (1967); Robbins et al., Diabetes 36:838-845 (1987); Cleland et al., Crit. Rev. Therapeutic Drug Carrier Systems 10:307-377 (1993)).

The replacement of amino acids can also change the selectivity of binding to cell surface receptors. Ostade et al., Nature 361:266-268 (1993) describes certain mutations resulting in selective binding of TNF-α to only one of the two known types of TNF receptors. Thus, the CAPP of the present invention may include one or more amino acid substitutions, deletions or additions, either from natural mutations or human manipulation.

As indicated, changes are preferably of a minor nature, such as conservative amino acid substitutions that do not significantly affect the folding or activity of the protein (see Table 1).

TABLE 1
Conservative Amino Acid Substitutions.

| | |
|---|---|
| Aromatic | Phenylalanine |
| | Tryptophan |
| | Tyrosine |
| Hydrophobic | Leucine |
| | Isoleucine |
| | Valine |
| Polar | Glutamine |
| | Asparagine |
| Basic | Arginine |
| | Lysine |
| | Histidine |
| Acidic | Aspartic Acid |
| | Glutamic Acid |
| Small | Alanine |
| | Serine |
| | Threonine |
| | Methionine |
| | Glycine |

Of course, the number of amino acid substitutions a skilled artisan would make depends on many factors, including those described above. Generally speaking, the number of amino acid substitutions for any given CAPP polypeptide will not be more than 50,40, 30, 20, 10, 5, or 3.

Amino acids in the CAPP protein of the present invention that are essential for function can be identified by methods known in the art, such as site-directed mutagenesis or alanine-scanning mutagenesis (Cunningham and Wells, Science 244:1081-1085 (1989)). The latter procedure introduces single alanine mutations at every residue in the molecule. The resulting mutant molecules are then tested for biological activity such as receptor binding or in vitro proliferative activity. Sites that are critical for ligand-receptor binding can also be determined by structural analysis such as crystallization, nuclear magnetic resonance or photoaffinity labeling (Smith et al., J. Mol. Biol. 224:899-904 (1992) and de Vos et al, Science 255:306-312 (1992)).

The polypeptides of the present invention are preferably provided in an isolated form. By “isolated polypeptide” is intended a polypeptide removed from its native environment. Thus, a polypeptide produced and/or contained within a recombinant host cell is considered isolated for purposes of the present invention. Also intended as an “isolated polypeptide” are polypeptides that have been purified, partially or substantially, from a recombinant host cell or from a native source. For example, a recombinantly produced version of the CAPP polypeptide can be substantially purified by the one-step method described in Smith and Johnson, Gene 67:31-40 (1988).

The polypeptides of the present invention include the polypeptide encoded by the deposited cDNA including the leader; the mature polypeptide encoded by the deposited the cDNA minus the leader (i.e., the mature protein); a polypeptide comprising amino acids about −32 to about 365 in SEQ ID NO:2; a polypeptide comprising amino acids about −31 to about 365 in SEQ ID NO:2; a polypeptide comprising amino acids about 1 to about 365 in SEQ ID NO:2; as well as polypeptides which have at least 90% similarity, more preferably at least 95% similarity, and still more preferably at least 96%, 97%, 98% or 99% similarity to those described above. Further polypeptides of the present invention include polypeptides at least 80% identical, more preferably at least 90% or 95% identical, still more preferably at least 96%, 97%, 98% or 99% identical to the polypeptide encoded by the deposited cDNA, to the polypeptide of FIG. 1 (SEQ ID NO:2), and also include portions of such polypeptides with at least 30 amino acids and more preferably at least 50 amino acids.

By “% similarity” for two polypeptides is intended a similarity score produced by comparing the amino acid sequences of the two polypeptides using the Bestfit program (Wisconsin Sequence Analysis Package, Version 8 for Unix, Genetics Computer Group, University Research Park, 575 Science Drive, Madison, Wis. 53711) and the default settings for determining similarity. Bestfit uses the local homology algorithm of Smith and Waterman (Advances in Applied Mathematics 2: 482-489, 1981) to find the best segment of similarity between two sequences.

By a polypeptide having an amino acid sequence at least, for example, 95% “identical” to a reference amino acid sequence of a CAPP polypeptide is intended that the amino acid sequence of the polypeptide is identical to the reference sequence except that the polypeptide sequence may include up to five amino acid alterations per each 100 amino acids of the reference amino acid of the CAPP polypeptide. In other words, to obtain a polypeptide having an amino acid sequence at least 95% identical to a reference amino acid sequence, up to 5% of the amino acid residues in the reference sequence may be deleted or substituted with another amino acid, or a number of amino acids up to 5% of the total amino acid residues in the reference sequence may be inserted into the reference sequence. These alterations of the reference sequence may occur at the amino or carboxy terminal positions of the reference amino acid sequence or anywhere between those terminal positions, interspersed either individually among residues in the reference sequence or in one or more contiguous groups within the reference sequence.

As a practical matter, whether any particular polypeptide is at least 90%, 95%, 96%, 97%, 98% or 99% identical to, for instance, the amino acid sequence shown in FIG. 1 (SEQ ID NO:2) or to the amino acid sequence encoded by deposited cDNA clone can be determined conventionally using known computer programs such the Bestfit program (Wisconsin Sequence Analysis Package, Version 8 for Unix, Genetics Computer Group, University Research Park, 575 Science Drive, Madison, Wis. 53711. When using Bestfit or any other sequence alignment program to determine whether a particular sequence is, for instance, 95% identical to a reference sequence according to the present invention, the parameters are set, of course, such that the percentage of identity is calculated over the full length of the reference amino acid sequence and that gaps in homology of up to 5% of the total number of amino acid residues in the reference sequence are allowed.

The polypeptide of the present invention could be used as a molecular weight marker on SDS-PAGE gels or on molecular sieve gel filtration columns using methods well known to those of skill in the art.

As described in detail below, the polypeptides of the present invention can also be used to raise polyclonal and monoclonal antibodies, which are useful in assays for detecting CAPP protein expression as described below or as agonists and antagonists capable of enhancing or inhibiting CAPP protein function. Further, such polypeptides can be used in the yeast two-hybrid system to “capture” CAPP protein binding proteins which are also candidate agonist and antagonist according to the present invention. The yeast two hybrid system is described in Fields and Song, Nature 340:245-246 (1989).

In another aspect, the invention provides a peptide or polypeptide comprising an epitope-bearing portion of a polypeptide of the invention. The epitope of this polypeptide portion is an immunogenic or antigenic epitope of a polypeptide of the invention. An “immunogenic epitope” is defined as a part of a protein that elicits an antibody response when the whole protein is the immunogen. These immunogenic epitopes are believed to be confined to a few loci on the molecule. On the other hand, a region of a protein molecule to which an antibody can bind is defined as an “antigenic epitope.” The number of immunogenic epitopes of a protein generally is less than the number of antigenic epitopes. See, for instance, Geysen et al., Proc. Natl. Acad Sci. USA 81:3998-4002 (1983).

As to the selection of peptides or polypeptides bearing an antigenic epitope (i.e., that contain a region of a protein molecule to which an antibody can bind), it is well known in that art that relatively short synthetic peptides that mimic part of a protein sequence are routinely capable of eliciting an antiserum that reacts with the partially mimicked protein. See, for instance, Sutcliffe, J. G. et al. “Antibodies that react with predetermined sites on proteins,” Science 219:660-666 (1983). Peptides capable of eliciting protein-reactive sera are frequently represented in the primary sequence of a protein, can be characterized by a set of simple chemical rules, and are confined neither to immunodominant regions of intact proteins (i.e., immunogenic epitopes) nor to the amino or carboxyl terminals. Peptides that are extremely hydrophobic and those of six or fewer residues generally are ineffective at inducing antibodies that bind to the mimicked protein; longer, peptides, especially those containing proline residues, usually are effective. Sutcliffe et al., supra, at 661. For instance, 18 of 20 peptides designed according to these guidelines, containing 8-39 residues covering 75% of the sequence of the influenza virus hemagglutinin HA1 polypeptide chain, induced antibodies that reacted with the HA1 protein or intact virus; and 12/12 peptides from the MuLV polymerase and 18/18 from the rabies glycoprotein induced antibodies that precipitated the respective proteins.

Antigenic epitope-bearing peptides and polypeptides of the invention are therefore useful to raise antibodies, including monoclonal antibodies, that bind specifically to a polypeptide of the invention. Thus, a high proportion of hybridomas obtained by fusion of spleen cells from donors immunized with an antigen epitope-bearing peptide generally secrete antibody reactive with the native protein. Sutcliffe et al., supra, at 663. The antibodies raised by antigenic epitope-bearing peptides or polypeptides are usefull to detect the mimicked protein, and antibodies to different peptides may be used for tracking the fate of various regions of a protein precursor which undergoes post-translational processing. The peptides and anti-peptide antibodies may be used in a variety of qualitative or quantitative assays for the mimicked protein, for instance in competition assays since it has been shown that even short peptides (e.g., about 9 amino acids) can bind and displace the larger peptides in immunoprecipitation assays. See, for instance, Wilson et al., Cell 37:767-778 (1984) at 777. The anti-peptide antibodies of the invention also are useful for purification of the mimicked protein, for instance, by adsorption chromatography using methods well known in the art.

Antigenic epitope-bearing peptides and polypeptides of the invention designed according to the above guidelines preferably contain a sequence of at least seven, more preferably at least nine and most preferably between about 15 to about 30 amino acids contained within the amino acid sequence of a polypeptide of the invention. However, peptides or polypeptides comprising a larger portion of an amino acid sequence of a polypeptide of the invention, containing about 30 to about 50 amino acids, or any length up to and including the entire amino acid sequence of a polypeptide of the invention, also are considered epitope-bearing peptides or polypeptides of the invention and also are useful for inducing antibodies that react with the mimicked protein. Preferably, the amino acid sequence of the epitope-bearing peptide is selected to provide substantial solubility in aqueous solvents (i.e., the sequence includes relatively hydrophilic residues and highly hydrophobic sequences are preferably avoided); and sequences containing proline residues are particularly preferred.

Non-limiting examples of antigenic polypeptides or peptides that can be used to generate CAPP-specific antibodies include: a polypeptide comprising amino acid residues from about −32 to about −22 in SEQ ID NO:2; a polypeptide comprising amino acid residues from about −4 to about 40 in SEQ ID NO:2; a polypeptide comprising amino acid residues from about 46 to about 57 in SEQ ID NO:2; a polypeptide comprising amino acid residues from about 62 to about 73 in SEQ ID NO:2 ; a polypeptide comprising amino acid residues from about 78 to about 87 in SEQ ID NO:2; a polypeptide comprising amino acid residues from about 92 to about 110 in SEQ ID NO:2 ; a polypeptide comprising amino acid residues from about 119 to about 144 in SEQ ID NO:2; a polypeptide comprising amino acid residues from about 152 to about 186 in SEQ ID NO:2; a polypeptide comprising amino acid residues from about 200 to about 219 in SEQ ID NO:2; a polypeptide comprising amino acid residues from about 230 to about 240 in SEQ ID NO:2; a polypeptide comprising amino acid residues from about 248 to about 258 in SEQ ID NO:2; a polypeptide comprising amino acid residues from about 314 to about 336 in SEQ ID NO:2; and a polypeptide comprising amino acid residues from about 344 to about 353 in SEQ ID NO:2. As indicated above, the inventors have determined that the above polypeptide fragments are antigenic regions of the CAPP protein.

The epitope-bearing peptides and polypeptides of the invention may be produced by any conventional means for making peptides or polypeptides including recombinant means using nucleic acid molecules of the invention. For instance, a short epitope-bearing amino acid sequence may be fused to a larger polypeptide which acts as a carrier during recombinant production and purification, as well as during immunization to produce anti-peptide antibodies. Epitope-bearing peptides also may be synthesized using known methods of chemical synthesis. For instance, Houghten has described a simple method for synthesis of large numbers of peptides, such as 10-20 mg of 248 different 13 residue peptides representing single amino acid variants of a segment of the HA1 polypeptide which were prepared and characterized (by ELISA-type binding studies) in less than four weeks. Houghten, R. A., “General method for the rapid solid-phase synthesis of large numbers of peptides: specificity of antigen-antibody interaction at the level of individual amino acids,” Proc. Natl. Acad. Sci. USA 82:5131-5135 (1985). This “Simultaneous Multiple Peptide Synthesis (SMPS)” process is further described in U.S. Pat. No. 4,631,211 to Houghten et al. (1986). In this procedure the individual resins for the solid-phase synthesis of various peptides are contained in separate solvent-permeable packets, enabling the optimal use of the many identical repetitive steps involved in solid-phase methods. A completely manual procedure allows 500-1000 or more syntheses to be conducted simultaneously. Houghten et al., supra, at 5134.

Epitope-bearing peptides and polypeptides of the invention are used to induce antibodies according to methods well known in the art. See, for instance, Sutcliffe et al., supra; Wilson et al., supra; Chow, M. et al., Proc. Natl. Acad. Sci. USA 82:910-914; and Bittle, F. J. et al., J. Gen. Virol. 66:2347-2354 (1985). Generally, animals may be immunized with free peptide; however, anti-peptide antibody titer may be boosted by coupling of the peptide to a macromolecular carrier, such as keyhole limpet hemacyanin (KLH) or tetanus toxoid. For instance, peptides containing cysteine may be coupled to carrier using a linker such as m-maleimidobenzoyl-N-hydroxysuccinimide ester (MBS), while other peptides may be coupled to carrier using a more general lining agent such as glutaraldehyde. Animals such as rabbits, rats and mice are immunized with either free or carrier-coupled peptides, for instance, by intraperitoneal and/or intradermal injection of emulsions containing about 100 mg peptide or carrier protein and Freund's adjuvant. Several booster injections may be needed, for instance, at intervals of about two weeks, to provide a useful titer of anti-peptide antibody which can be detected, for example, by ELISA assay using free peptide adsorbed to a solid surface. The titer of anti-peptide antibodies in serum from an immunized animal may be increased by selection of anti-peptide antibodies, for instance, by adsorption to the peptide on a solid support and elution of the selected antibodies according to methods well known in the art.

Immunogenic epitope-bearing peptides of the invention, i.e., those parts of a protein that elicit an antibody response when the whole protein is the immunogen, are identified according to methods known in the art. For instance, Geysen et al., supra, discloses a procedure for rapid concurrent synthesis on solid supports of hundreds of peptides of sufficient purity to react in an enzyme-linked immunosorbent assay. Interaction of synthesized peptides with antibodies is then easily detected without removing them from the support. In this manner a peptide bearing an immunogenic epitope of a desired protein may be identified routinely by one of ordinary skill in the art. For instance, the immunologically important epitope in the coat protein of foot-and-mouth disease virus was located by Geysen et al. with a resolution of seven amino acids by synthesis of an overlapping set of all 208 possible hexapeptides covering the entire 213 amino acid sequence of the protein. Then, a complete replacement set of peptides in which all 20 amino acids were substituted in turn at every position within the epitope were synthesized, and the particular amino acids conferring specificity for the reaction with antibody were determined. Thus, peptide analogs of the epitope-bearing peptides of the invention can be made routinely by this method. U.S. Pat. No. 4,708,781 to Geysen (1987) further describes this method of identifying a peptide bearing an immunogenic epitope of a desired protein.

Further still, U.S. Pat. No. 5,194,392 to Geysen (1990) describes a general method of detecting or determining the sequence of monomers (amino acids or other compounds) which is a topological equivalent of the epitope (i.e., a “mimotope”) which is complementary to a particular paratope (antigen binding site) of an antibody of interest. More generally, U.S. Pat. No. 4,433,092 to Geysen (1989) describes a method of detecting or determining a sequence of monomers which is atopographical equivalent of a ligand which is complementary to the ligand binding site of a particular receptor of interest. Similarly, U.S. Pat. No. 5,480,971 to Houghten, R. A. et al. (1996) on “Peralkylated Oligopeptide Mixtures” discloses linear C₁-C₇-alkyl peralkylated oligopeptides and sets and libraries of such peptides, as well as methods for using such oligopeptide sets and libraries for determining the sequence of a peralkylated oligopeptide that preferentially binds to an acceptor molecule of interest. Thus, non-peptide analogs of the epitope-bearing peptides of the invention also can be made routinely by these methods.

The entire disclosure of each document cited in this section on “Polypeptides and Peptides” is hereby incorporated herein by reference.

As one of skill in the art will appreciate, CAPP polypeptides of the present invention and the epitope-bearing fragments thereof described above can be combined with parts of the constant domain of immunoglobulins (IgG), resulting in chimeric polypeptides. These fusion proteins facilitate purification and show an increased half-life in vivo. This has been shown, e.g., for chimeric proteins consisting of the first two domains of the human CD4-polypeptide and various domains of the constant regions of the heavy or light chains of mammalian immunoglobulins (EP A 394,827; Traunecker et al., Nature 331:84-86 (1988)). Fusion proteins that have a disulfide-linked dimeric structure due to the IgG part can also be more efficient in binding and neutralizing other molecules than the monomeric CAPP protein or protein fragment alone (Fountoulakis et al, J. Biochem 270:3958-3964 (1995)).

### Disease State Diagnosis and Prognosis

It is believed that certain maladies in mammals may cause the mammals to express significantly altered levels of the CAPP protein and mRNA encoding the CAPP protein when compared to a corresponding “standard” mammal, i.e., a mammal of the same species not having the malady or condition. For example, a mammal suffering from pancreatitis or a condition that causes abnormal myocardial hypertrophy is expected to express altered levels of CAPP by the pancreas or heart, respectively. Further, it is believed that decreased levels of the CAPP protein can be detected in certain body fluids (e.g., sera, plasma, urine, and spinal fluid) from mammals with such a condition when compared to sera from mammals of the same species not having the condition. Thus, the invention provides a diagnostic method useful during diagnosis or pancreatitis or one of the many conditions that cause abnormal hypertrophy of the heart, such as hypertension, myocardial infarction, valve disease and cardiomyopathy. The method involves assaying the expression level of the gene encoding the CAPP protein in mammalian cells or body fluid and comparing the gene expression level with a standard CAPP gene expression level, whereby a decrease in the gene expression level over the standard is indicative of said conditions.

Where a diagnosis has already been made according to conventional methods, the present invention is useful as a prognostic indicator, whereby patients exhibiting decreased CAPP gene expression will experience a worse clinical outcome relative to patients expressing the gene at a lower level.

Additionally, the presence of CAPP protein or mRNA level can be measured to qualitatively determine cell or tissue type. Since CAPP is highly expressed in mature heart, pancreas and placenta tissue, CAPP expression can be employed to determine the type of cells that are present in a cell culture.

The CAPP gene was discovered in an activated T-cell cDNA library. CAPP protein and mRNA expression can be used as a marker to detect activated T-cells. Monitoring T cells activation is usefull for a number of in vitro diagnostic purposes, including studying the effects of candidate drugs on the immune system, and determining whether the T cells of a subject have been activated by analyzing a blood sample taken from the subject or by assessing activity in an in vitro screening test.

By “assaying the expression level of the gene encoding the CAPP protein” is intended qualitatively or quantitatively measuring or estimating the level of the CAPP protein or the level of the mRNA encoding the CAPP protein in a first biological sample either directly (e.g., by determining or estimating absolute protein level or mRNA level) or relatively (e.g., by comparing to the CAPP protein level or mRNA level in a second biological sample).

Preferably, the CAPP protein level or mRNA level in the first biological sample is measured or estimated and compared to a standard CAPP protein level or mRNA level, the standard being taken from a second biological sample obtained from an individual not having the cancer. As will be appreciated in the art, once a standard CAPP protein level or mRNA level is known, it can be used repeatedly as a standard for comparison.

By “biological sample” is intended any biological sample obtained from an individual, cell line, tissue culture, or other source which contains CAPP protein or mRNA. Biological samples include mammalian body fluids (such as sera, plasma, urine, synovial fluid and spinal fluid) which contain secreted mature CAPP protein, and heart, placenta, pancreas and umbilical tissue. Methods for obtaining tissue biopsies and body fluids from mammals are well known in the art. Where the biological sample is to include mRNA, a tissue biopsy is the preferred source.

Preferred mammals include monkeys, apes, cats, dogs, cows, pigs, horses, rabbits and humans. Particularly preferred are humans.

Total cellular RNA can be isolated from a biological sample using any suitable technique such as the single-step guanidinium-thiocyanate-phenol-chloroform method described in Chomczynski and Sacchi, Anal. Biochem. 162:156-159 (1987). Levels of mRNA encoding the CAPP protein are then assayed using any appropriate method. These include Northern blot analysis, S1 nuclease mapping, the polymerase chain reaction (PCR), reverse transcription in combination with the polymerase chain reaction (RT-PCR), and reverse transcription in combination with the ligase chain reaction (RT-LCR).

Northern blot analysis can be performed as described in Harada et al., Cell 63:303-312 (1990). Briefly, total RNA is prepared from a biological sample as described above. For the Northern blot, the RNA is denatured in an appropriate buffer (such as glyoxal/dimethyl sulfoxide/sodium phosphate buffer), subjected to agarose gel electrophoresis, and transferred onto a nitrocellulose filter. After the RNAs have been linked to the filter by a UV linker, the filter is prehybridized in a solution containing formamide, SSC, Denhardt's solution, denatured salmon sperm, SDS, and sodium phosphate buffer. CAPP protein cDNA labeled according to any appropriate method (such as the ³²P-multiprimed DNA labeling system (Amersham)) is used as probe. After hybridization overnight, the filter is washed and exposed to x-ray film. cDNA for use as probe according to the present invention is described in the sections above and will preferably at least 15 bp in length.

S1 mapping can be performed as described in Fujita et al., Cell 49:357-367 (1987). To prepare probe DNA for use in SI mapping, the sense strand of above-described cDNA is used as a template to synthesize labeled antisense DNA. The antisense DNA can then be digested using an appropriate restriction endonuclease to generate further DNA probes of a desired length. Such antisense probes are useful for visualizing protected bands corresponding to the target mRNA (i.e., mRNA encoding the CAPP protein). Northern blot analysis can be performed as described above.

Preferably, levels of mRNA encoding the CAPP protein are assayed using the RT-PCR method described in Makino et al., Technique 2:295-301 (1990). By this method, the radioactivities of the “amplicons” in the polyacrylamide gel bands are linearly related to the initial concentration of the target mRNA. Briefly, this method involves adding total RNA isolated from a biological sample in a reaction mixture containing a RT primer and appropriate buffer. After incubating for primer annealing, the mixture can be supplemented with a RT buffer, dNTPs, DTT, RNase inhibitor and reverse transcriptase. After incubation to achieve reverse transcription of the RNA, the RT products are then subject to PCR using labeled primers. Alternatively, rather than labeling the primers, a labeled DNTP can be included in the PCR reaction mixture. PCR amplification can be performed in a DNA thermal cycler according to conventional techniques. After a suitable number of rounds to achieve amplification, the PCR reaction mixture is electrophoresed on a polyacrylamide gel. After drying the gel, the radioactivity of the appropriate bands (corresponding to the mRNA encoding the CAPP protein)) is quantified using an imaging analyzer. RT and PCR reaction ingredients and conditions, reagent and gel concentrations, and labeling methods are well known in the art. Variations on the RT-PCR method will be apparent to the skilled artisan.

Any set of oligonucleotide primers which will amplify reverse transcribed target mRNA can be used and can be designed as described in the sections above.

Assaying CAPP protein levels in a biological sample can occur using any art-known method. Preferred for assaying CAPP protein levels in a biological sample are antibody-based techniques. For example, CAPP protein expression in tissues can be studied with classical immunohistological methods. In these, the specific recognition is provided by the primary antibody (polyclonal or monoclonal) but the secondary detection system can utilize fluorescent, enzyme, or other conjugated secondary antibodies. As a result, an immunohistological staining of tissue section for pathological examination is obtained. Tissues can also be extracted, e.g., with urea and neutral detergent, for the liberation of CAPP protein for Western-blot or dot/slot assay (Jalkanen, M., et al., J. Cell. Biol. 101:976-985 (1985); Jalkanen, M., et al., J. Cell. Biol. 105:3087-3096 (1987)). In this technique, which is based on the use of cationic solid phases, quantitation of CAPP protein can be accomplished using isolated CAPP protein as a standard. This technique can also be applied to body fluids. With these samples, a molar concentration of CAPP protein will aid to set standard values of CAPP protein content for different body fluids, like serum, plasma, urine, spinal fluid, etc. The normal appearance of CAPP protein amounts can then be set using values from healthy individuals, which can be compared to those obtained from a test subject.

Other antibody-based methods useful for detecting CAPP protein gene expression include immunoassays, such as the enzyme linked immunosorbent assay (ELISA) and the radioimmunoassay (RIA). For example, a CAPP protein-specific monoclonal antibodies can be used both as an immunoabsorbent and as an enzyme-labeled probe to detect and quantify the CAPP protein. The amount of CAPP protein present in the sample can be calculated by reference to the amount present in a standard preparation using a linear regression computer algorithm. Such an ELISA for detecting a tumor antigen is described in lacobelli et al., Breast Cancer Research and Treatment 11:19-30 (1988). In another ELISA assay, two distinct specific monoclonal antibodies can be used to detect CAPP protein in a body fluid. In this assay, one of the antibodies is used as the immunoabsorbent and the other as the enzyme-labeled probe.

The above techniques may be conducted essentially as a “one-step” or “two-step” assay. The “one-step” assay involves contacting CAPP protein with immobilized antibody and, without washing, contacting the mixture with the labeled antibody. The “two-step” assay involves washing before contacting the mixture with the labeled antibody. Other conventional methods may also be employed as suitable. It is usually desirable to immobilize one component of the assay system on a support, thereby allowing other components of the system to be brought into contact with the component and readily removed from the sample.

Suitable enzyme labels include, for example, those from the oxidase group, which catalyze the production of hydrogen peroxide by reacting with substrate. Glucose oxidase is particularly preferred as it has good stability and its substrate (glucose) is readily available. Activity of an oxidase label may be assayed by measuring the concentration of hydrogen peroxide formed by the enzyme-labelled antibody/substrate reaction. Besides enzymes, other suitable labels include radioisotopes, such as iodine (¹²⁵I, ¹²¹I), carbon (¹⁴C), sulfur (³⁵S), tritium (3H), indium (¹¹¹In), and technetium (^{99m}Tc), and fluorescent labels, such as fluorescein and rhodamine, and biotin.

In addition to assaying CAPP protein levels in abiological sample obtained from an individual, CAPP protein can also be detected in vivo by imaging. Antibody labels or markers for in vivo imaging of CAPP protein include those detectable by X-radiography, NMR or ESR. For X-radiography, suitable labels include radioisotopes such as barium or caesium, which emit detectable radiation but are not overtly harmful to the subject. Suitable markers for NMR and ESR include those with a detectable characteristic spin, such as deuterium, which may be incorporated into the antibody by labeling of nutrients for the relevant hybridoma.

A CAPP protein-specific antibody or antibody fragment which has been labeled with an appropriate detectable imaging moiety, such as a radioisotope (for example, ¹³¹I, ¹¹¹In, ^{99m}Tc), a radio-opaque substance, or a material detectable by nuclear magnetic resonance, is introduced (for example, parenterally, subcutaneously or intraperitoneally) into the mammal to be examined for cancer. It will be understood in the art that the size of the subject and the imaging system used will determine the quantity of imaging moiety needed to produce diagnostic images. In the case of a radioisotope moiety, for a human subject, the quantity of radioactivity injected will normally range from about 5 to 20 millicuries of ^{99m}Tc. The labeled antibody or antibody fragment will then preferentially accumulate at the location of cells which contain CAPP protein. In vivo tumor imaging is described in S. W. Burchiel et al., “Immunopharmacokinetics of Radiolabelled Antibodies and Their Fragments” (Chapter 13 in Tumor Imaging: The Radiochemical Detection of Cancer, eds., S. W. Burchiel and B. A. Rhodes, Masson Publishing Inc. (1982)).

CAPP-protein specific antibodies for use in the present invention can be raised against the intact CAPP protein or an antigenic polypeptide fragment thereof, which may presented together with a carrier protein, such as an albumin, to an animal system (such as rabbit or mouse) or, if it is long enough (at least about 25 amino acids), without a carrier.

As used herein, the term “antibody” (Ab) or “monoclonal antibody” (Mab) is meant to include intact molecules as well as antibody fragments (such as, for example, Fab and F(ab′)₂ fragments) which are capable of specifically binding to CAPP protein. Fab and F(ab′)₂ fragments lack the Fc fragment of intact antibody, clear more rapidly from the circulation, and may have less non-specific tissue binding of an intact antibody (Wahl et al., J. Nucl. Med. 24:316-325 (1983)). Thus, these fragments are preferred.

The antibodies of the present invention may be prepared by any of a variety of methods. For example, cells expressing the CAPP protein or an antigenic fragment thereof can be administered to an animal in order to induce the production of sera containing polyclonal antibodies. In a preferred method, a preparation of CAPP protein is prepared and purified to render it substantially free of natural contaminants. Such a preparation is then introduced into an animal in order to produce polyclonal antisera of greater specific activity.

In the most preferred method, the antibodies of the present invention are monoclonal antibodies (or CAPP protein binding fragments thereof). Such monoclonal antibodies can be prepared using hybridoma technology (Kohler et al., Nature 256:495 (1975); Kohler et al., Eur. J. Immunol. 6:511 (1976); Kohler et al., Eur. J. Immunol. 6:292 (1976); Hammerling et al., In: Monoclonal Antibodies and T-Cell Hybridomas, Elsevier, N.Y., pp. 563-681 (1981)). In general, such procedures involve immunizing an animal (preferably a mouse) with a CAPP protein antigen or, more preferably, with a CAPP protein-expressing cell. Suitable cells can be recognized by their capacity to bind anti-CAPP protein antibody. Such cells may be cultured in any suitable tissue culture medium; however, it is preferable to culture cells in Earle's modified Eagle's medium supplemented with 10% fetal bovine serum (inactivated at about 56° C.), and supplemented with about 10 g/l of nonessential amino acids, about 1,000 U/ml of penicillin, and about 100 g/ml of streptomycin. The splenocytes of such mice are extracted and fused with a suitable myeloma cell line. Any suitable myeloma cell line may be employed in accordance with the present invention; however, it is preferable to employ the parent myeloma cell line (SP₂O₂), available from the American Type Culture Collection, Rockville, Md. After fusion, the resulting hybridoma cells are selectively maintained in HAT medium, and then cloned by limiting dilution as described by Wands et al. (Gastroenterology 80:225-232 (1981)). The hybridoma cells obtained through such a selection are then assayed to identify clones which secrete antibodies capable of binding the CAPP protein antigen.

Alternatively, additional antibodies capable of binding to the CAPP protein antigen may be produced in a two-step procedure through the use of anti-idiotypic antibodies. Such a method makes use of the fact that antibodies are themselves antigens, and that, therefore, it is possible to obtain an antibody which binds to a second antibody. In accordance with this method, CAPP-protein specific antibodies are used to immunize an animal, preferably a mouse. The splenocytes of such an animal are then used to produce hybridoma cells, and the hybridoma cells are screened to identify clones which produce an antibody whose ability to bind to the CAPP protein-specific antibody can be blocked by the CAPP protein antigen. Such antibodies comprise anti-idiotypic antibodies to the CAPP protein-specific antibody and can be used to immunize an animal to induce formation of further CAPP protein-specific antibodies.

It will be appreciated that Fab and F(ab′)₂ and other fragments of the antibodies of the present invention may be used according to the methods disclosed herein. Such fragments are typically produced by proteolytic cleavage, using enzymes such as papain (to produce Fab fragments) or pepsin (to produce F(ab′)₂ fragments). Alternatively, CAPP protein-binding fragments can be produced through the application of recombinant DNA technology or through synthetic chemistry.

Where in vivo imaging is used to detect enhanced levels of CAPP protein for tumor diagnosis in humans, it may be preferable to use “humanized” chimeric monoclonal antibodies. Such antibodies can be produced using genetic constructs derived from hybridoma cells producing the monoclonal antibodies described above. Methods for producing chimeric antibodies are known in the art. See, for review, Morrison, Science 229:1202 (1985); Oi et al, BioTechniques 4:214 (1986); Cabilly et al, U.S. Pat. No. 4,816,567; Taniguchi et al., EP 171496; Morrison et al., EP 173494; Neuberger et al., WO 8601533; Robinson et al., WO 8702671; Boulianne et al., Nature 312:643 (1984); Neuberger et al., Nature 314:268 (1985).

Further suitable labels for the CAPP protein-specific antibodies of the present invention are provided below. Examples of suitable enzyme labels include malate dehydrogenase, staphylococcal nuclease, delta-5-steroid isomerase, yeast-alcohol dehydrogenase, alpha-glycerol phosphate dehydrogenase, triose phosphate isomerase, peroxidase, alkaline phosphatase, asparaginase, glucose oxidase, beta-galactosidase, ribonuclease, urease, catalase, glucose-6-phosphate dehydrogenase, glucoamylase, and acetylcholine esterase.

Examples of suitable radioisotopic labels include ³H, ¹¹¹In, ¹²⁵I, ¹³¹I, ³²P, ³⁵S, ¹⁴C, ⁵¹Cr, ⁵⁷To, ⁵⁸Co, ⁵⁹Fe, ⁷⁵Se, ¹⁵²Eu, ⁹⁰Y, ⁶⁷Cu, ²¹⁷Ci, ²¹¹At, ²¹²Pb, ⁴⁷Sc, ¹⁰⁹Pd, ⁹⁹mTc, etc. ¹¹¹In and ^{99m}Tc are preferred isotopes where in vivo imaging is used since its avoids the problem of dehalogenation of the ¹²⁵I or ¹³¹I-labeled monoclonal antibody by the liver. In addition, this radionucleotide has a more favorable gamma emission energy for imaging (Perkins et al., Eur. J. Nucl. Med. 10:296-301 (1985); Carasquillo et al., J. Nucl. Med. 28:281-287 (1987)). For example, ¹¹¹In coupled to monoclonal antibodies with 1-(P-isothiocyanatobenzyl)-DPTA has shown little uptake in non-tumorous tissues, particularly the liver, and therefore enhances specificity of tumor localization (Esteban et al., J. Nucl. Med 28:861-870 (1987)).

Examples of suitable non-radioactive isotopic labels include ¹⁵⁷Gd, ⁵⁵Mn, ¹⁶²Dy, ⁵²Tr, and ⁵⁶Fe, preferably chelated with a complexing ligand suitable for in vivo use.

Examples of suitable fluorescent labels include an ¹⁵²Eu label, a fluorescein label, an isothiocyanate label, a rhodamine label, a phycoerythrin, label, a phycocyanin label, an allophycocyanin label, an o-phthaldehyde label, and a fluorescamine label.

Examples of suitable toxin labels include diphtheria toxin, ricin, and cholera toxin.

Examples of chemiluminescent labels include aluminal label, an isoluminal label, an aromatic acridinium ester label, an imidazole label, an acridinium salt label, an oxalate ester label, a luciferin label, a luciferase label, and an aequorin label.

Examples of nuclear magnetic resonance contrasting agents include heavy metal nuclei such as Gd, Mn, and iron, preferably chelated with a complexing ligand suitable for in vivo use.

Typical techniques for binding the above-described labels to antibodies are provided by Kennedy et al., Clin. Chim. Acta 70:1-31 (1976), and Schurs et al, Clin. Chim. Acta 81:1-40 (1977). Coupling techniques mentioned in the latter are the glutaraldehyde method, the periodate method, the dimaleimide method, the m-maleimidobenzyl-N-hydroxy-succinimide ester method, all of which methods are incorporated by reference herein.

### Therapeutics and Use in Cell Culture

The inventors contemplate that the CAPP polypeptide functions as a growth factor or similar cellular signaling polypeptide in vivo. CAPP possesses homology to the Drosophila brainiac polypeptide. See, FIG. 2. This polypeptide is a neurogenic secreted molecule that is believed to play a role in the differentiation of embryonic cells into neurons. Thus, it is contemplated that the CAPP polypeptide exerts an effect on the differentiation of cells in the early stages of cell and tissue development, and may serve to aid in the differentiation of embryonic cells into heart or pancreas cells.

The CAPP polypeptide is also highly expressed in adult heart and pancreas tissue. One role of CAPP in mature muscle tissue may be to inhibit cell replication and division in the mature muscle tissue.

Thus, the inventors contemplate a number of additional practical utilities that use the growth-effecting properties of the CAPP polypeptide to module the differentiation and proliferation of cells and tissue, both in vivo and ex vivo.

Assessing the modulating effects of the CAPP polypeptide on the cellular proliferation and differentiation of cells can be performed as described below. Biological activity of CAPP polypeptides can be examined in organ culture assays or in colony assay systems in agarose culture. Stimulation or inhibition of cellular proliferation may be measured by a variety of assays. For observing cell growth inhibition, one can use a solid or liquid medium. In a solid medium, cells undergoing growth inhibition can easily be selected from the subject cell group by comparing the sizes of colonies formed. In a liquid medium, growth inhibition can be screened by measuring culture broth turbity or incorporation of labeled thymidine into DNA. Typically, the incorporation of a nucleoside analog into newly synthesized DNA is employed to measure proliferation (active cell growth) in a population of cells. For example, bromodeoxyuridine (BrdU) can be employed as a DNA labeling reagent and Anti-BrdU mouse monoclonal antibody (clone BMC 9318 IgG₁) can be employed as a detection reagent. This antibody binds only to cells containing DNA which has incorporated bromodeoxyuridine. A number of detection methods can be used in conjunction with this assay including immunofluorescence, immunohistochemical, ELISA and colorimetric methods. Kits that include bromodeoxyuridine (BrdU) and Anti-BrdU mouse monoclonal antibody are commercially available from Boehringer Mannheim (Indianapolis, Ind.).

Effect upon cellular differentiation can be measured by contacting embryonic cells with various amounts of a CAPP polypeptide and observing the effect upon differentiation of the embryonic cells. Tissue specific antibodies and microscopy may be used to identify the resulting cells.

### Modes of Administration

It will be appreciated that conditions caused by a decrease in the standard or normal level of CAPP activity in an individual, can be treated by administration of CAPP protein. Thus, the invention further provides a method of treating an individual in need of an increased level of CAPP activity comprising administering to such an individual a pharmaceutical composition comprising an effective amount of an isolated CAPP polypeptide of the invention, particularly a mature form of the CAPP, effective to increase the CAPP activity level in such an individual.

The CAPP polypeptide composition will be formulated and dosed in a fashion consistent with good medical practice, taking into account the clinical condition of the individual patient (especially the side effects of treatment with CAPP polypeptide alone), the site of delivery of the CAPP polypeptide composition, the method of administration, the scheduling of administration, and other factors known to practitioners. The “effective amount” of CAPP polypeptide for purposes herein is thus determined by such considerations.

As a general proposition, the total pharmaceutically effective amount of CAPP polypeptide administered parenterally per dose will be in the range of about 1 μg/kg/day to 10 mg/kg/day of patient body weight, although, as noted above, this will be subject to therapeutic discretion. More preferably, this dose is at least 0.01 mg/kg/day, and most preferably for humans between about 0.01 and 1 mg/kg/day for the hormone. If given continuously, the CAPP polypeptide is typically administered at a dose rate of about 1 μg/kg/hour to about 50 μg/kg/hour, either by 1-4 injections per day or by continuous subcutaneous infusions, for example, using a mini-pump. An intravenous bag solution may also be employed. The length of treatment needed to observe changes and the interval following treatment for responses to occur appears to vary depending on the desired effect.

Pharmaceutical compositions containing the CAPP of the invention may be administered orally, rectally, parenterally, intracistemally, intravaginally, intraperitoneally, topically (as by powders, ointments, drops or transdermal patch), bucally, or as an oral or nasal spray. By “pharmaceutically acceptable carrier” is meant a non-toxic solid, semisolid or liquid filler, diluent, encapsulating material or formulation auxiliary of any type. The term “parenteral” as used herein refers to modes of administration which include intravenous, intramuscular, intraperitoneal, intrastemal, subcutaneous and intraaticular injection and infusion.

The CAPP polypeptide is also suitably administered by sustained-release systems. Suitable examples of sustained-release compositions include semi-permeable polymer matrices in the form of shaped articles, e.g., films, or microcapsules. Sustained-release matrices include polylactides (U.S. Pat. No. 3,773,919, EP 58,481), copolymers of L-glutamic acid and gamma-ethyl-L-glutamate (Sidman, U. et al., Biopolymers 22:547-556 (1983)), poly (2-hydroxyethyl methacrylate) (R. Langer et al., J. Biomed. Mater. Res. 15:167-277 (1981), and R. Langer, Chem. Tech. 12:98-105 (1982)), ethylene vinyl acetate (R. Langer et al., Id) or poly-D-(−)-3-hydroxybutyric acid (EP 133,988). Sustained-release CAPP polypeptide compositions also include liposomally entrapped CAPP polypeptide. Liposomes containing CAPP polypeptide are prepared by methods known per se: DE 3,218,121; Epstein et al., Proc. Natl. Acad. Sci. (USA) 82:3688-3692 (1985); Hwang et al, Proc. Natl. Acad. Sci. (USA) 77:4030-4034 (1980); EP 52,322; EP 36,676; EP 88,046; EP 143,949; EP 142,641; Japanese Pat. Appl. 83-118008; U.S. Pat. Nos. 4,485,045 and 4,544,545; and EP 102,324. Ordinarily, the liposomes are of the small (about 200-800 Angstroms) unilamellar type in which the lipid content is greater than about 30 mol. percent cholesterol, the selected proportion being adjusted for the optimal CAPP polypeptide therapy.

For parenteral administration, in one embodiment, the CAPP polypeptide is formulated generally by mixing it at the desired degree of purity, in a unit dosage injectable form (solution, suspension, or emulsion), with a pharmaceutically acceptable carrier, i.e., one that is non-toxic to recipients at the dosages and concentrations employed and is compatible with other ingredients of the formulation. For example, the formulation preferably does not include oxidizing agents and other compounds that are known to be deleterious to polypeptides.

Generally, the formulations are prepared by contacting the CAPP polypeptide uniformly and intimately with liquid carriers or finely divided solid carriers or both. Then, if necessary, the product is shaped into the desired formulation. Preferably the carrier is a parenteral carrier, more preferably a solution that is isotonic with the blood of the recipient. Examples of such carrier vehicles include water, saline, Ringer's solution, and dextrose solution. Non-aqueous vehicles such as fixed oils and ethyl oleate are also useful herein, as well as liposomes.

The carrier suitably contains minor amounts of additives such as substances that enhance isotonicity and chemical stability. Such materials are non-toxic to recipients at the dosages and concentrations employed, and include buffers such as phosphate, citrate, succinate, acetic acid, and other organic acids or their salts; antioxidants such as ascorbic acid; low molecular weight (less than about ten residues) polypeptides, e.g., polyarginine or tripeptides; proteins, such as serum albumin, gelatin, or immunoglobulins; hydrophilic polymers such as polyvinylpyrrolidone; amino acids, such as glycine, glutamic acid, aspartic acid, or arginine; monosaccharides, disaccharides, and other carbohydrates including cellulose or its derivatives, glucose, mannose, or dextrins; chelating agents such as EDTA; sugar alcohols such as mannitol or sorbitol; counterions such as sodium; and/or nonionic surfactants such as polysorbates, poloxamers, or PEG.

The CAPP polypeptide is typically formulated in such vehicles at a concentration of about 0.1 mg/ml to 100 mg/ml, preferably 1-10 mg/ml, at a pH of about 3 to 8. It will be understood that the use of certain of the foregoing excipients, carriers, or stabilizers will result in the formation of CAPP polypeptide salts.

CAPP polypeptide to be used for therapeutic administration must be sterile. Sterility is readily accomplished by filtration through sterile filtration membranes (e.g., 0.2 micron membranes). Therapeutic CAPP polypeptide compositions generally are placed into a container having a sterile access port, for example, an intravenous solution bag or vial having a stopper pierceable by a hypodermic injection needle.

CAPP polypeptide ordinarily will be stored in unit or multi-dose containers, for example, sealed ampoules or vials, as an aqueous solution or as a lyophilized formulation for reconstitution. As an example of a lyophilized formulation, 10-ml vials are filled with 5 ml of sterile-filtered 1% (w/v) aqueous CAPP polypeptide solution, and the resulting mixture is lyophilized. The infusion solution is prepared by reconstituting the lyophilized CAPP polypeptide using bacteriostatic Water-for-Injection.

The invention also provides a pharmaceutical pack or kit comprising one or more containers filled with one or more of the ingredients of the pharmaceutical compositions of the invention. Associated with such container(s) can be a notice in the form prescribed by a governmental agency regulating the manufacture, use or sale of pharmaceuticals or biological products, which notice reflects approval by the agency of manufacture, use or sale for human administration. In addition, the polypeptides of the present invention may be employed in conjunction with other therapeutic compounds.

For use in cell culture media, the CAPP polypeptide can be added to a culture medium to aid in the differentiation and maintenance of cultured heart, pancreas and placenta cells. Useful concentration ranges for this purpose are from about 10 picograms/mL to about 10 micrograms/mL.

### Chromosome Assays

The nucleic acid molecules of the present invention are also valuable for chromosome identification. The sequence is specifically targeted to and can hybridize with a particular location on an individual human chromosome. Moreover, there is a current need for identifying particular sites on the chromosome. Few chromosome marking reagents based on actual sequence data (repeat polymorphisms) are presently available for marking chromosomal location. The mapping of DNAs to chromosomes according to the present invention is an important first step in correlating those sequences with genes associated with disease.

In certain preferred embodiments in this regard, the cDNA herein disclosed is used to clone genomic DNA of a CAPP protein gene. This can be accomplished using a variety of well known techniques and libraries, which generally are available commercially. The genomic DNA then is used for in situ chromosome mapping using well known techniques for this purpose. Typically, in accordance with routine procedures for chromosome mapping, some trial and error may be necessary to identify a genomic probe that gives a good in situ hybridization signal.

In addition, in some cases, sequences can be mapped to chromosomes by preparing PCR primers (preferably 15-25 bp) from the cDNA. Computer analysis of the 3′ untranslated region of the gene is used to rapidly select primers that do not span more than one exon in the genomic DNA, thus complicating the amplification process. These primers are then used for PCR screening of somatic cell hybrids containing individual human chromosomes. Only those hybrids containing the human gene corresponding to the primer will yield an amplified portion.

PCR mapping of somatic cell hybrids is a rapid procedure for assigning a particular DNA to a particular chromosome. Using the present invention with the same oligonucleotide primers, sublocalization can be achieved with panels of portions from specific chromosomes or pools of large genomic clones in an analogous manner. Other mapping strategies that can similarly be used to map to its chromosome include in situ hybridization, prescreening with labeled flow-sorted chromosomes and preselection by hybridization to construct chromosome specific-cDNA libraries.

Fluorescence in situ hybridization (“FISH”) of a cDNA clone to a metaphase chromosomal spread can be used to provide a precise chromosomal location in one step. This technique can be used with probes from the cDNA as short as 50 or 60 bp. For a review of this technique, see Verma et al., Human Chromosomes: A Manual Of Basic Techniques, Pergamon Press, New York (1988).

Once a sequence has been mapped to a precise chromosomal location, the physical position of the sequence on the chromosome can be correlated with genetic map data. Such data are found, for example, in V. McKusick, Mendelian Inheritance In Man, available on-line through Johns Hopkins University, Welch Medical Library. The relationship between genes and diseases that have been mapped to the same chromosomal region are then identified through linkage analysis (coinheritance of physically adjacent genes).

Next, it is necessary to determine the differences in the cDNA or genomic sequence between affected and unaffected individuals. If a mutation is observed in some or all of the affected individuals but not in any normal individuals, then the mutation is likely to be the causative agent of the disease.

With current resolution of physical mapping and genetic mapping techniques, a cDNA precisely localized to a chromosomal region associated with the disease could be one of between 50 and 500 potential causative genes. This assumes 1 megabase mapping resolution and one gene per 20 kb.

Having generally described the invention, the same will be more readily understood by reference to the following examples, which are provided by way of illustration and are not intended as limiting.

### EXAMPLES

Example 1

### Expression and Purification of CAPP protein in E. coli

The DNA sequence encoding the mature CAPP protein in the deposited cDNA clone is amplified using PCR oligonucleotide primers specific to the amino terminal sequences of the CAPP protein and to vector sequences 3′ to the gene. Additional nucleotides containing restriction sites to facilitate cloning are added to the 5′ and 3′ sequences respectively.

The 5′ oligonucleotide primer has the sequence:

5′ AGCA GGATCC CAA GAA AAA AAT GGA AAA GGG 3 ′ (SEQ ID NO:4) containing the underlined BamHI restriction site, which encodes 21 nucleotides of coding sequence in FIG. 1 (SEQ ID NO:1) beginning with Q after the S in the signal peptide.

The 3′ primer has the sequence:

5′ ATTG TCTAGA TAT CTA TTT TAG CAT TTT A 3 ′ (SEQ ID NO:5) containing the underlined XbaI restriction site followed by 19 nucleotides of sequence, including the last 8 nucleotides of the coding domain in FIG. 1.

The restriction sites are convenient to restriction enzyme sites in the bacterial expression vector pQE9, which are used for bacterial expression in these examples. (Qiagen, Inc. 9259 Eton Avenue, Chatsworth, Calif., 91311). pQE9 encodes ampicirin antibiotic resistance (“Amp”) and contains a bacterial origin of replication (“ori”), an IPTG inducible promoter, a ribosome binding site (“RBS”), a 6-His tag and restriction enzyme sites.

The amplified CAPP DNA and the vector pQE9 both are digested with BamHI and XbaI and the digested DNAs are then ligated together. Insertion of the CAPP protein DNA into the restricted pQE9 vector places the CAPP protein coding region downstream of and operably linked to the vector's IPTG-inducible promoter and in-frame with an initiating AUG appropriately positioned for translation of CAPP protein.

The ligation mixture is transformed into competent E. coli cells using standard procedures. Such procedures are described in Sambrook et al., Molecular Cloning: a Laboratory Manual, 2nd Ed.; Cold Spring Harbor Laboratory Press, Cold Spring Harbor, N.Y. (1989). E. coli strain MR15/rep4, containing multiple copies of the plasmid pREP4, which expresses lac repressor and confers kanamycin resistance (“Kan^{r}”), is used in carrying out the illustrative example described herein. This strain, which is only one of many that are suitable for expressing CAPP protein, is available commercially from Qiagen.

Transformants are identified by their ability to grow on LB plates in the presence of ampicillin and kanamycin. Plasmid DNA is isolated from resistant colonies and the identity of the cloned DNA confirmed by restriction analysis.

Clones containing the desired constructs are grown overnight (“ON”) in liquid culture in LB media supplemented with both ampicillin (100 μg/ml) and kanamycin (25 μg/ml).

The ON culture is used to inoculate a large culture, at a dilution of approximately 1:100 to 1:250. The cells are grown to an optical density at 600 nm (“OD600”) of between 0.4 and 0.6. Isopropyl-B-D-thiogalactopyranoside (“IPTG”) is then added to a final concentration of 1 mM to induce transcription from lac repressor sensitive promoters, by inactivating the lacI repressor. Cells subsequently are incubated further for 3 to 4 hours. Cells then are harvested by centrifugation and disrupted, by standard methods. Inclusion bodies are purified from the disrupted cells using routine collection techniques, and protein is solubilized from the inclusion bodies into 8M urea. The 8M urea solution containing the solubilized protein is passed over aPD-10 column in2×phosphate-buffered saline (“PBS”), thereby removing the urea, exchanging the buffer and refolding the protein. The protein is purified by a further step of chromatography to remove endotoxin. Then, it is sterile filtered. The sterile filtered protein preparation is stored in 2×PBS at a concentration of 95 μ/ml.

### Example 2

### Cloning and Expression of CAPP protein in a Baculovirus Expression System

The DNA sequence encoding the full length human CAPP protein, ATCC #97729, is amplified using PCR oligonucleotide primers corresponding to the 5′ and 3′ sequences of the gene as follows.

The 5′ primer has the sequence:

5 ′ GGCC GGATCC GCC ATC ATG AGT OTT GGA COT CGA AGA AT 3′ (SEQ ID NO:6) containing the underlined BamHI restriction enzyme site followed by 6 nucleotides resembling an efficient signal for the initiation of translation in eukaryotic cells (Kozak, M., J. Mol. Biol. 196:947-950 (1987)), followed by 23 nucleotides of coding sequence of the human CAPP gene (the initiation codon for translation “ATG” is double underlined).

The 3′ primer has the sequence:

5′ ATTG TCTAGA TAT CTA TTT TAG CAT TTT A 3′ (SEQ ID NO:5) containing the underlined XbaI restriction site followed by 19 nucleotides of CAPP sequence, including the last 8 nucleotides of the coding domain.

The amplified sequences are isolated from a 1% agarose gel using a commercially available kit (“Geneclean,” BIO 101 Inc., La Jolla, Calif.). The fragment is then digested with the endonucleases BamHI and XbaI and then purified again on a 1% agarose gel. This fragment is designated F2.

The vector A2 (modification of pVL941 vector, discussed below) is used for expression of the human CAPP protein using the baculoviris expression system (see, Summers, M. D. and Smith, G. E. 1987, a Manual of Methods for Baculovirus Vectors and Insect Cell Culture Procedures, Texas Agricultural Experimental Station Bulletin No. 1555). This expression vector contains the strong polyhedrin promoter of the Autographa californica nuclear polyhidrosis virus (AcMNPV) followed by the recognition sites for the restriction endonucleases BamHI and Asp718. The polyadenylation site of the simian virus (SV)40 is used for efficient polyadenylation. For an easy selection of recombinant viruses the beta-galactosidase gene from E. coli is inserted in the same orientation as the polyhedrin promoter followed by the polyadenylation signal of the polyhedrin gene. The polyhedrin sequences are flanked on both sides by viral sequences for the cell-mediated homologous recombination of cotransfected wild-type viral DNA. Many other baculovirus vectors can be used in place of A2 such as pAc373, pVL941 and pAcIM1 (Luckow, V. A. and Summers, M. D., Virology, 170:31-39).

The plasmid is digested with the restriction enzymes XbaI and BamHI and then dephosphorylated using calf intestinal phosphatase by procedures known in the art. The DNA is then isolated from a 1% agarose gel using the commercially available kit (“Geneclean” BIO 101 Inc., La Jolla, Calif.). This vector DNA is designated V2.

Fragment F2 and the dephosphorylated plasmid V2 are ligated with T4 DNA ligase. E. coli HB101 cells are then transformed and bacteria identified that contained the plasmid (pBac CAPP) with the CAPP gene using the PCR method, in which one of the primers is that used to amplify the gene and the second primer is from well within the vector so that only those bacterial colonies containing the CAPP gene fragment will show amplification of the DNA.. The sequence of the cloned fragment is confirmed by DNA sequencing.

5 ug of the plasmid pBac CAPP is cotransfected with 1 ug of a commercially available linearized baculovirus (“BaculoGold baculovirus DNA”, Pharmingen, San Diego, Calif.) using the lipofection method (Felgner et al. Proc. Natl. Acad Sci. USA, 84:7413-7417 (1987)).

1 ug of BaculoGold virus DNA and 5 ug of the plasmid pBac CAPP are mixed in a sterile well of a microtiter plate containing 50 μl of serum free Grace's medium (Life Technologies Inc., Gaithersburg, Md.). Afterwards 10 μl Lipofectin plus 90 μl Grace's medium are added, mixed and incubated for 15 minutes at room temperature. Then the transfection mixture is added dropwise to the Sf9 insect cells (ATCC CRL 1711) seeded in a 35 mm tissue culture plate with 1 ml Grace's medium without serum. The plate is rocked back and forth to mix the newly added solution. The plate is then incubated for 5 hours at 27° C. After 5 hours the transfection solution is removed from the plate and 1 ml of Grace's insect medium supplemented with 10% fetal calf serum is added. The plate is put back into an incubator and cultivation continued at 27° C. for four days.

After four days the supernatant is collected and a plaque assay performed similar to that described by Summers and Smith (supra). As a modification, an agarose gel with “Blue Gal” (Life Technologies Inc., Gaithersburg) is used which allows an easy isolation of blue stained plaques. (A detailed description of a “plaque assay” can also be found in the user's guide for insect cell culture and baculovirology distributed by Life Technologies Inc., Gaithersburg, page 9-10).

Four days after a serial dilution of the viruses is added to the cells, blue stained plaques are picked with the tip of an Pasteur pipette. The agar containing the recombinant viruses is then resuspended in an Eppendorf tube containing 200 μl of Grace's medium. The agar is removed by a brief centrifugation and the supernatant containing the recombinant baculoviruses (designated baculovirus V-CAPP) is used to infect Sf9 cells seeded in 35 mm dishes. Four days later the supernatants of these culture dishes are harvested and then stored at 4° C.

Sf9 cells are grown in Grace's medium supplemented with 10% heat-inactivated FBS. The cells are infected with the recombinant baculovirus V-CAPP at a multiplicity of infection (MOI) of 2. Six hours later the medium is removed and replaced with SF900 II medium minus methionine and cysteine (Life Technologies Inc., Gaithersburg). 42 hours later 5 uCi of ³⁵S-methionine and 5 uCi ³⁵S cysteine (Amersham) are added. The cells are further incubated for 16 hours before they are harvested by centrifugation and the labelled proteins visualized by SDS-PAGE and autoradiography.

### Example 3

### Cloning and Expression in Mammalian Cells

Most of the vectors used for the transient expression of the CAPP protein gene sequence in mammalian cells should carry the SV40 origin of replication. This allows the replication of the vector to high copy numbers in cells (e.g. COS cells) which express the T antigen required for the initiation of viral DNA synthesis. Any other mammalian cell line can also be utilized for this purpose.

A typical mammalian expression vector contains the promoter element, which mediates the initiation of transcription of mRNA, the protein coding sequence, and signals required for the termination of transcription and polyadenylation of the transcript. Additional elements include enhancers, Kozak sequences and intervening sequences flanked by donor and acceptor sites for RNA splicing. Highly efficient transcription can be achieved with the early and late promoters from SV40, the long terminal repeats (LTRs) from Retroviruses, e.g. RSV, HTLVI, HIVI and the early promoter of the cytomegalovirus (CMV). However, cellular signals can also be used (e.g. human actin promoter). Suitable expression vectors for use in practicing the present invention include, for example, vectors such as pSVL and pMSG (Pharmacia, Uppsala, Sweden), pRSVcat (ATCC 37152), pSV2dhfr (ATCC 37146) and pBC12MI (ATCC 67109). Mammalian host cells that could be used include, human Hela, 283, H9 and Jurkart cells, mouse NIH3T3 and C127 cells, Cos 1, Cos 7 and CV1, African green monkey cells, quail QC1-3 cells, mouse L cells and Chinese hamster ovary cells.

Alternatively, the gene can be expressed in stable cell lines that contain the gene integrated into a chromosome. The co-transfection with a selectable marker such as dhfr, gpt, neomycin, hygromycin allows the identification and isolation of the transfected cells.

The transfected gene can also be amplified to express large amounts of the encoded protein. The DHFR (dihydrofolate reductase) is a useful marker to develop cell lines that carry several hundred or even several thousand copies of the gene of interest. Another useful selection marker is the enzyme glutamine synthase (GS) (Murphy et al., Biochem J. 227:277-279 (1991); Bebbington et al., Bio/Technology 10:169-175 (1992)). Using these markers, the mammalian cells are grown in selective medium and the cells with the highest resistance are selected. These cell lines contain the amplified gene(s) integrated into a chromosome. Chinese hamster ovary (CHO) cells are often used for the production of proteins.

The expression vectors pC1 and pC4 contain the strong promoter (LTR) of the Rous Sarcoma Virus (Cullen et al., Molecular and Cellular Biology, 438-447 (March, 1985)) plus a fragment of the CMV-enhancer (Boshart et al., Cell 41:521-530(1985)). Multiple cloning sites, e.g. with the restriction enzyme cleavage sites BamHI, XbaI and Asp718, facilitate the cloning of the gene of interest. The vectors contain in addition the 3′ intron, the polyadenylation and termination signal of the rat preproinsulin gene.

### Example 3(a)

### Cloning and Expression of CAPP in COS7 Cells

The expression of plasmid, human CAPP HA is derived from a vector pCDNA3 (Invitrogen) containing: 1) SV40 origin of replication, 2) ampicillin resistance gene, 3) E.coli replication origin, 4) CMV promoter followed by a polylinker region, a SV40 intron and polyadenylation site. A DNA fragment encoding the entire human CAPP precursor and a HA tag fused in frame to its 3′ end is cloned into the polylinker region of the vector, therefore, the recombinant protein expression is directed by the CMV promoter. The HA tag corresponds to an epitope derived from the influenza hemagglutinin protein as previously described (I. Wilson et al., Cell 37:767 (1984)). The inclusion of HA tag to our target protein allows easy detection of the recombinant protein with an antibody that recognizes the HA epitope.

The plasmid construction strategy is described as follows. The DNA sequence encoding human CAPP, ATCC #97729, is constructed by PCR on the original clone using two primers.

Suitable primers include the following, which are used in this example.

The 5′ primer has the sequence:

5′ GGCC GGATCC GCC ATC ATG AGT GTT GGA COT CGA AGA AT 3′ (SEQ ID NO:6) containing the underlined BamHI restriction enzyme site followed by a Kozak sequence, followed by 23 nucleotides of coding sequence of CAPP protein in FIG. 1. Inserted into an expression vector, as described below, the 5′ end of the amplified fragment encoding CAPP provides an efficient signal peptide.

The 3′ primer has the sequence:

| | |
|---|---|
| 5′ ATTGTCTAGA ATT TTA AGC GTA GTC TGG GAC GTC GTA | (SEQ ID NO:7) |
| TGG GTA GCA TTT TAA ATG AGC ACT CTG 3′ | |

containing the underlined XbaI restriction site followed by translation stop codon, HA tag and nucleotides of CAPP sequence, including nucleotides of the coding domain set out in FIG. 1.

Therefore, the PCR product contains a BamHI site, human CAPP coding sequence followed by HA tag fused in frame, a translation termination stop codon next to the HA tag, and a XbaI site. The PCR amplified DNA fragment is digested with the restriction enzyme BamHI and XbaI and the vector, pcDNA3 is digested with BamHI restriction enzyme and ligated. The ligation mixture is transformed into E. coli strain SURE (available from Stratagene Cloning Systems, 11099 North Torrey Pines Road, La Jolla, Calif. 92037). The transformed culture is plated on ampicillin media plates and resistant colonies are selected. Plasmid DNA is isolated from transformants and examined by restriction analysis for the presence of the correct fragment.

For expression of the recombinant CAPP, COS cells are transfected with the expression vector by DEAE-DEXTRAN method. (J. Sambrook, E. Fritsch, T. Maniatis, Molecular Cloning: A Laboratory Manual, Cold Spring Laboratory Press, (1989)). Expression of the CAPP-HA protein is detected by radiolabelling and immunoprecipitation method. (E. Harlow, D. Lane, Antibodies: A Laboratory Manual, Cold Spring Harbor Laboratory Press, (1988)). Cells are labelled for 8 hours wit ³⁵S-cysteine two days post transfection. Culture media are then collected and cells are lysed with detergent (RIPA buffer (150 mM NaCl, 1% NP40, 0.1 SDS, 1 NP-40, 0. DOC, 50 mM Tris, pH 7.5). (Wilson, I. et al., Cell 37:767 (1984)). Both cell lysate and culture media are precipitated with a HA specific monoclonal antibody. Proteins precipitated are analyzed on 15 SDS-PAGE gels.

### Example 3(b)

### Cloning and Expression in CHO Cells

The vector pC4 is used for the expression of CAPP protein. Plasmid pC4 is a derivative of the plasmid pSV2-dhfr [ATCC Accession No. 37146]. The plasmid contains the mouse DHFR gene under control of the SV40 early promoter. Chinese hamster ovary or other cells lacking dihydrofolate activity that are transfected with these plasmids can be selected by growing the cells in a selective medium (alpha minus MEM, Life Technologies) supplemented with the chemotherapeutic agent methotrexate. The amplification of the DHFR genes in cells resistant to methotrexate (MTX) has been well documented (see, e.g., Alt, F. W., Kellems, R. M., Bertino, J. R., and Schimke, R. T., J. Biol. Chem. 253:1357-1370 (1978), Hamlin, J. L. and Ma, C., Biochim. et Biophys. Act, 1097:107-143 (1990), Page, M. J. and Sydenham, M. A., Biotechnology 9:64-68 (1991)). Cells grown in increasing concentrations of MTX develop resistance to the drug by overproducing the target enzyme, DHFR, as a result of amplification of the DHFR gene. If a second gene is linked to the DHFR gene it is usually co-amplified and over-expressed. It is state of the art to develop cell lines carrying more than 1,000 copies of the genes. Subsequently, when the methotrexate is withdrawn cell lines contain the amplified gene integrated into the chromosome(s).

Plasmid pC4 contains for the expression of the gene of interest a strong promoter of the long terminal repeat (LTR) of the Rouse Sarcoma Virus (Cullen et al, Molecular and Cellular Biology, March 1985, 438-4470) plus a fragment isolated from the enhancer of the immediate early gene of human cytomegalovirus (CMV) (Boshart et al., Cell 41:521-530, (1985)). Downstream of the promoter is a restriction enzyme site that allows the integration of a gene of interest. Behind the cloning site the plasmid contains translational stop codons in all three reading frames followed by the 3′ intron and the polyadenylation site of the rat preproinsulin gene. Other high efficient promoters can also be used for the expression, e.g., the human β-actin promoter, the SV40 early or late promoters or the long terminal repeats from other retroviruses, e.g., HIV and HTLVI. For the polyadenylation of the mRNA other signals, e.g., from the human growth hormone or globin genes can be used as well.

Stable cell lines carrying a gene of interest integrated into the chromosomes can also be selected upon co-transfection with a selectable marker such as gpt, G418 or hygromycin. It is advantageous to use more than one selectable marker in the beginning, e.g. G418 plus methotrexate.

The plasmid pC4 is digested with the restriction enzymes BamHI and XbaI and then dephosphorylated using calf intestinal phosphates by procedures known in the art. The vector is then isolated from a 1% agarose gel.

The DNA sequence encoding CAPP, ATCC 97729, is amplified using PCR oligonucleotide primers corresponding to the 5′ and 3′ sequences of the gene:

The 5′ primer has the sequence:

5′ GGCC GGATCC GCC ATC ATG AGT GTT GGA CGT CGA AGA AT 3′ (SEQ ID NO:6) containing the underlined BamHI restriction enzyme site followed by an optimized Kozak sequence for insect cell expression, followed by 23 nucleotides of coding sequence of CAPP protein in FIG. 1. Inserted into an expression vector, as described below, the 5′ end of the amplified fragment encoding CAPP provides an efficient signal peptide. An efficient signal for initiation of translation in eukaryotic cells, as described by Kozak, M., J. Mol. Biol. 196: 947-950 (1987) is appropriately located in the vector portion of the construct.

The 3′ primer has the sequence:

| | |
|---|---|
| 5′ ATTGTCTAGA ATT TTA AGC GTA GTC TGG GAC GTC GTA | (SEQ ID NO:7) |
| TGG GTA GCA TTT TAA ATG AGC ACT CTG 3′ | |

containing the underlined XbaI restriction site followed by translation stop codon, HA tag and nucleotides of CAPP sequence, including nucleotides of the coding domain set out in FIG. 1.

The amplified fragments are isolated from a 1% agarose gel as described above and then digested with the endonucleases BamHI and XbaI and then purified again on a 1% agarose gel.

The isolated fragment and the dephosphorylated vector are then ligated with T4 DNA ligase. E.coli HB101 cells are then transformed and bacteria identified that contained the plasmid pC4 inserted in the correct orientation using the restriction enzyme BamHI. The sequence of the inserted gene is confirmed by DNA sequencing.

### Transfection of CHO-DHFR-cells

Chinese hamster ovary cells lacking an active DHFR enzyme are used for transfection. 5 μg of the expression plasmid C4 are cotransfected with 0.5 μg of the plasmid pSVneo using the lipofecting method (Felgner et al., supra). The plasmid pSV2-neo contains a dominant selectable marker, the gene neo from Tn5 encoding an enzyme that confers resistance to a group of antibiotics including G418. The cells are seeded in alpha minus MEM supplemented with 1 mg/ml G418. After 2 days, the cells are trypsinized and seeded in hybridoma cloning plates (Greiner, Germany) and cultivated from 10-14 days. After this period, single clones are trypsinized and then seeded in 6-well petri dishes using different concentrations of methotrexate (25 nM, 50 nM, 100 nM, 200 nM, 400 nM). Clones growing at the highest concentrations of methotrexate are then transferred to new 6-well plates containing even higher concentrations of methotrexate (500 nM, 1 μM, 2 μM, 5 μM). The same procedure is repeated until clones grow at a concentration of 100 μM.

The expression of the desired gene product is analyzed by Western blot analysis and SDS-PAGE.

### Example 4

### Tissue Distribution of CAPP Protein Expression

Northern blot analysis is carried out to examine CAPP gene expression in human tissues, using methods described by, among others, Sambrook et al., cited above. A cDNA probe containing the entire nucleotide sequence of the CAPP protein (SEQ ID NO:1) is labeled with 32P using the rediprime™ DNA labeling system (Amersham Life Science), according to manufacturer's instructions. After labelling, the probe is purified using a CHROMA SPIN-100™ column (Clontech Laboratories, Inc.), according to manufacturer's protocol number PT1200-1. The purified labelled probe is then used to examine various human tissues for CAPP mRNA.

Multiple Tissue Northern (MTN) blots containing various human tissues (H) or human immune system tissues (IM) are obtained from Clontech and are examined with labelled probe using ExpressHyb™ hybridization solution (Clontech) according to manufacturer's protocol number PT1190-1. Following hybridization and washing, the blots are mounted and exposed to film at −70° C. overnight, and films developed according to standard procedures. By Northern blot analysis it has been determined that this gene is abundant in adult heart and pancreas, with low amounts in placenta, lung, liver, skeletal muscle, kidney, spleen, thymus, prostate, testis, ovary, small intestine, colon and peripheral blood leukocytes. The gene was identified by database distribution in activated T cells (3), CD34 positive cells, Ntera2 cells 14 days after RA stimulation, kidney cortex, adult heart, Jurkat cells and small intestine.

It will be clear that the invention may be practiced otherwise than as particularly described in the foregoing description and examples.

Numerous modifications and variations of the present invention are possible in light of the above teachings and, therefore, are within the scope of the appended claims.

The entire disclosure of all publications (including patents, patent applications, journal articles, laboratory manuals, books, or other documents) cited herein are hereby incorporated by reference.

What is claimed is:

1. An isolated nucleic acid molecule comprising a polynucleotide having a nucleotide sequence at least 95% identical to a sequence selected from the group consisting of:
(a) a nucleotide sequence encoding a polypeptide comprising amino acids from about −32 to about 365 in SEQ ID NO:2;
(b) a nucleotide sequence encoding a polypeptide comprising amino acids from about −31 to about 365 in SEQ ID NO:2;
(c) a nucleotide sequence encoding a polypeptide comprising amino acids from about 1 to about 365 in SEQ ID NO:2;
(d) a nucleotide sequence encoding a polypeptide having the amino acid sequence encoded by the cDNA clone contained in ATCC Deposit No. 97729; and
(e) a nucleotide sequence complementary to any of the nucleotide sequences in (a), (b), (c) or (d).

2. An isolated nucleic acid molecule comprising a polynucleotide which encodes the amino acid sequence of an epitope-bearing portion of an CAPP polypeptide having an amino acid sequence in (a), (b), (c) or (d) of claim 1.

3. The isolated nucleic acid molecule of claim 1, which encodes an epitope-bearing portion of a CAPP polypeptide selected from the group consisting of: a polypeptide comprising amino acid residues from about −32 to about −22 in SEQ ID NO:2; a polypeptide comprising amino acid residues from about −4 to about 40 in SEQ ID NO:2; a polypeptide comprising amino acid residues from about 46 to about 57 in SEQ ID NO:2; a polypeptide comprising amino acid residues from about 62 to about 73 in SEQ ID NO:2; a polypeptide comprising amino acid residues from about 78 to about 87 in SEQ ID NO:2; a polypeptide comprising amino acid residues from about 92 to about 110 in SEQ ID NO:2; a polypeptide comprising amino acid residues from about 119 to about 144 in SEQ ID NO:2; a polypeptide comprising amino acid residues from about 152 to about 186 in SEQ ID NO:2; a polypeptide comprising amino acid residues from about 200 to about 219 in SEQ ID NO:2; a polypeptide comprising amino acid residues from about 230 to about 240 in SEQ ID NO:2; a polypeptide comprising amino acid residues from about 248 to about 258 in SEQ ID NO:2; a polypeptide comprising amino acid residues from about 314 to about 336 in SEQ ID NO:2; and a polypeptide comprising amino acid residues from about 344 to about 353 in SEQ ID NO:2.

4. An isolated nucleic acid molecule, comprising a polynucleotide having a sequence selected from the group consisting of:
(a) the nucleotide sequence of a fragment of the sequence shown in SEQ ID NO:1, wherein said fragment comprises at least 50 contiguous nucleotides of SEQ ID NO:1; and
(b) a nucleotide sequence complementary to a nucleotide sequence in (a).

5. A method for making a recombinant vector comprising inserting an isolated nucleic acid molecule of claim 1 into a vector.

6. A recombinant vector produced by the method of claim 5.

7. A method of making a recombinant host cell comprising introducing the recombinant vector of claim 6 into a host cell.

8. A recombinant host cell produced by the method of claim 7.

9. A recombinant method for producing any of the CAPP polypeptides, comprising culturing the recombinant host cell of claim 8 under conditions such that said polypeptide is expressed and recovering said polypeptide.

10. An isolated CAPP polypeptide having an amino acid sequence at least 95% identical to a sequence selected from the group consisting of:
(a) amino acids from about −32 to about 365 in SEQ ID NO:2;
(b) amino acids from about −31 to about 365 in SEQ ID NO:2;
(c) amino acids from about 1 to about 365 in SEQ ID NO:2;
(d) the amino acid sequence of the CAPP polypeptide having the amino acid sequence encoded by the cDNA clones contained in ATCC Deposit No. 97729; and
(e) the amino acid sequence of an epitope-bearing portion of any one of the polypeptides of (a), (b), (c) or (d).

11. An isolated polypeptide comprising an epitope-bearing portion of the CAPP protein, wherein said portion is selected from the group consisting of: a polypeptide comprising amino acid residues from about −32 to about −22 in SEQ ID NO:2; a polypeptide comprising amino acid residues from about −4 to about 40 in SEQ ID NO:2; a polypeptide comprising amino acid residues from about 46 to about 57 in SEQ ID NO:2 ; a polypeptide comprising amino acid residues from about 62 to about 73 in SEQ ID NO:2 ; a polypeptide comprising amino acid residues from about 78 to about 87 in SEQ ID NO:2; a polypeptide comprising amino acid residues from about 92 to about 110 in SEQ ID NO:2; a polypeptide comprising amino acid residues from about 119 to about 144 in SEQ ID NO:2; a polypeptide comprising amino acid residues from about 152 to about 186 in SEQ ID NO:2; a polypeptide comprising amino acid residues from about 200 to about 219 in SEQ ID NO:2; a polypeptide comprising amino acid residues from about 230 to about 240 in SEQ ID NO:2; a polypeptide comprising amino acid residues from about 248 to about 258 in SEQ ID NO:2; a polypeptide comprising amino acid residues from about 314 to about 336 in SEQ ID NO:2; and a polypeptide comprising amino acid residues from about 344 to about 353 in SEQ ID NO:2.

12. An isolated antibody that binds specifically to a CAPP polypeptide of claim 10.

13. An isolated nucleic acid molecule comprising a polynucleotide encoding a CAPP polypeptide wherein, except for one to fifty conservative amino acid substitutions, said polypeptide has a sequence selected from the group consisting of:
(a) a nucleotide sequence encoding a polypeptide comprising amino acids from about −32 to about 365 in SEQ ID NO:2;
(b) a nucleotide sequence encoding a polypeptide comprising amino acids from about −31 to about 365 in SEQ ID NO:2;
(c) a nucleotide sequence encoding a polypeptide comprising amino acids from about 1 to about 365 in SEQ ID NO:2;
(d) a nucleotide sequence encoding a polypeptide having the amino acid sequence encoded by the cDNA clone contained in ATCC Deposit No. 97729; and
(e) a nucleotide sequence complementary to any of the nucleotide sequences in (a), (b), (c) or (d).

14. An isolated CAPP polypeptide wherein, except for at least one conservative amino acid substitution, said polypeptide has a sequence selected from the group consisting of:
(a) amino acids from about −32 to about 365 in SEQ ID NO:2;
(b) amino acids from about −31 to about 365 in SEQ ID NO:2;
(c) amino acids from about 1 to about 365 in SEQ ID NO:2;
(d) the amino acid sequence of the CAPP polypeptide having the amino acid sequence encoded by the cDNA clone contained in ATCC Deposit No. 97729; and
(e) the amino acid sequence of an epitope-bearing portion of any one of the polypeptides of (a), (b), (c) or (d).

15. An isolated antibody that binds specifically to a CAPP polypeptide of claim 10.


SEQUENCE LISTING (42 sequences)

SEQ ID NO: 1 - DNA (genomic), 2745 bp; double-stranded, linear
  CDS 233..1423
  sig_peptide 233..328
  mat_peptide 329..1423
gcagcggcacggcagcagcggcaacaagtgccggactagcagagccaagccggagcagtccctgccgccgacaccgccgggccgcccgtccggggcgccgcgcatggagcgtgagctgcggcggtcgccggggctgagccgcgcggagcgccgggacgtggatgtggccgcgatctcccgcccttgcccccgccccgccgagctggagctgctcccggacaagatatgagaaatgagtgttggacgtcgaagaataaagttgttgggtatcctgatgatggcaaatgtcttcatttattttattatggaagtctccaaaagcagtagccaagaaaaaaatggaaaaggggaagtaataatacccaaagagaagttctggaagatatctacccctcccgaggcatactggaaccgagagcaagagaagctgaaccggcagtacaaccccatcctgagcatgctgaccaaccagacgggggaggcgggcaggctctccaatataagccatctgaactactgcgaacctgacctgagggtcacgtcggtggttacgggttttaacaacttgccggacagatttaaagactttctgctgtatttgagatgccgcaattattcactgcttatagatcagccggataagtgtgcaaagaaacctttcttgttgctggcgattaagtccctcactccacattttgccagaaggcaagcaatccgggaatcctggggccaagaaagcaacgcagggaaccaaacggtggtgcgagtcttcctgctgggccagacacccccagaggacaaccaccccgacctttcagatatgctgaaatttgagagtgagaagcaccaagacattcttatgtggaactacagagacactttcttcaacttgtctctgaaggaagtgctgtttctcaggtgggtaagtacttcctgcccagacactgagtttgttttcaagggcgatgacgatgtttttgtgaacacccatcacatcctgaattacttgaatagtttatccaagaccaaagccaaagatctcttcataggtgatgtgatccacaatgctggacctcatcgggataagaagctgaagtactacatcccagaagttgtttactctggcctctacccaccctatgcagggggaggggggttcctctactccggccacctggccctgaggctgtaccatatcactgaccaggtccatctctaccccattgatgacgtttatactggaatgtgccttcagaaactcggcctcgttccagagaaacacaaaggcttcaggacatttgatatcgaggagaaaaacaaaaataacatctgctcctatgtagatctgatgttagtacatagtagaaaacctcaagagatgattgatatttggtctcagttgcagagtgctcatttaaaatgctaaaatagatacaaactcaattttgcatagaaaggtgtattttgaatagttcccatgttgtgttctcacattagagtaatttctatattaaaccatgaaaattgcctttatgagtgatacccatttgagggcctctaaacccttcaatttggtactcacgtgaagagggaaagcggaagatggtaatttttttttatggatgatatggcaggatgattggttctgatcttaccggctagtggtcatttttaaaaaacttgtaccctcttatctgaaatcctgtttctggaatttggccattttaagtgattttgtttgccctcttctataatattcctacttcccataataatgactgatttatttgtaattcaggtatttataaacctattggctacaaagactttgttaaacattatccagtggttttcgtgaaatggaattatgtttatttttatgggatttgggtaaattttaaattgtctagaaaactgaaatttcagttgtcagttgtggaattcagtttttcaattgtggaaatttcctgccaccccaacagtatttttgtgtgttaattaattttgcaaaatgagaatcatggtgtgacactcatctaatttatcttgttgtgatgttatggtcataataaggagaaagagggtttaatttttcttgtatttggtttcctggtggtatcatagtgtaattttagtatttgaaaatcagtgtgattccttaatggccaactgaagattgaattgccgctaacaaccatatcgtgttagtgaattttcaatatggaccaggaaggcatatgtattttgaacttgagtgaaaaggttgaagttacagacttttgcatagatggtttgtcaatttaaaattccagaatttattattgccatattttcacatgctgcttatacaagattattattgagtagtaactgttccctgtctatgtagaagtgcctgtgtttttatttattgttccagatcaaagaccaaaacattttcttaaatatctcttatgtaatattttatttgtatacagtgttgttgatgaaatatttaactagagcatgatattttaaatgttaaggtgtaacatatgttaaataaaactgttatttttgaattttaaaatttgttttttgggggtatgaactactagagtttaaaattctgccaaactattacttatatgtactattgtgtaacatactttcttgaaatatttttgtttatagaattgaaggttcttatcagatgggatactggggattataaacaatggaaataaagccactgtatttttaaaa

SEQ ID NO: 2 - protein, 397 aa
MSVGRRRIKLLGILMMANVFIYFIMEVSKSSSQEKNGKGEVIIPKEKFWKISTPPEAYWNREQEKLNRQYNPILSMLTNQTGEAGRLSNISHLNYCEPDLRVTSVVTGFNNLPDRFKDFLLYLRCRNYSLLIDQPDKCAKKPFLLLAIKSLTPHFARRQAIRESWGQESNAGNQTVVRVFLLGQTPPEDNHPDLSDMLKFESEKHQDILMWNYRDTFFNLSLKEVLFLRWVSTSCPDTEFVFKGDDDVFVNTHHILNYLNSLSKTKAKDLFIGDVIHNAGPHRDKKLKYYIPEVVYSGLYPPYAGGGGFLYSGHLALRLYHITDQVHLYPIDDVYTGMCLQKLGLVPEKHKGFRTFDIEEKNKNNICSYVDLMLVHSRKPQEMIDIWSQLQSAHLKC

SEQ ID NO: 3 - protein, 323 aa
QSKHRKLLLRCLLVLPLILLVDYCGLLTHLHELNFERHFHYPLNDDTGSGSASSGLDKFAYLRVPSFTAEVPVDQPARLTMLIKSAVGNSRRREAIRRTWGYEGRFSDVHLRRVFLLGTAEDSEKDVAWESREHGDILQADFTDAYFNNTLKTMLGMRWASEQFNRSEFYLFVDDDYYVSAKNVLKFLGRGRQSHQPELLFAGHVFQTSPLRHKFSKWYVSLEEYPFDRWPPYVTAGAFILSQKALRQLYAASVHLPLFRFDDVYLGIVALKAGISLQHCDDFRFHRPAYKGPDSYSSVIASHEFGDPEEMTRVWNECRSANY

SEQ ID NO: 4 - DNA (cDNA), 31 bp; single-stranded, linear
agcaggatcccaagaaaaaaatggaaaaggg

SEQ ID NO: 5 - DNA (cDNA), 29 bp; single-stranded, linear
attgtctagatatctattttagcatttta

SEQ ID NO: 6 - DNA (cDNA), 39 bp; single-stranded, linear
ggccggatccgccatcatgagtgttggacgtcgaagaat

SEQ ID NO: 7 - DNA (cDNA), 64 bp; single-stranded, linear
attgtctagaattttaagcgtagtctgggacgtcgtatgggtagcattttaaatgagcactctg

SEQ ID NO: 8 - DNA (cDNA), 3974 bp; both-stranded, both
ggtacctaagtgagtagggcgtccgatcgacggacgccttttttttgaattcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcgtcgacaattcgcgcgcgaaggcgaagcggcatgcatttacgttgacaccatcgaatggtgcaaaacctttcgcggtatggcatgatagcgcccggaagagagtcaattcagggtggtgaatgtgaaaccagtaacgttatacgatgtcgcagagtatgccggtgtctcttatcagaccgtttcccgcgtggtgaaccaggccagccacgtttctgcgaaaacgcgggaaaaagtggaagcggcgatggcggagctgaattacattcccaaccgcgtggcacaacaactggcgggcaaacagtcgttgctgattggcgttgccacctccagtctggccctgcacgcgccgtcgcaaattgtcgcggcgattaaatctcgcgccgatcaactgggtgccagcgtggtggtgtcgatggtagaacgaagcggcgtcgaagcctgtaaagcggcggtgcacaatcttctcgcgcaacgcgtcagtgggctgatcattaactatccgctggatgaccaggatgccattgctgtggaagctgcctgcactaatgttccggcgttatttcttgatgtctctgaccagacacccatcaacagtattattttctcccatgaagacggtacgcgactgggcgtggagcatctggtcgcattgggtcaccagcaaatcgcgctgttagcgggcccattaagttctgtctcggcgcgtctgcgtctggctggctggcataaatatctcactcgcaatcaaattcagccgatagcggaacgggaaggcgactggagtgccatgtccggttttcaacaaaccatgcaaatgctgaatgagggcatcgttcccactgcgatgctggttgccaacgatcagatggcgctgggcgcaatgcgcgccattaccgagtccgggctgcgcgttggtgcggatatctcggtagtgggatacgacgataccgaagacagctcatgttatatcccgccgttaaccaccatcaaacaggattttcgcctgctggggcaaaccagcgtggaccgcttgctgcaactctctcagggccaggcggtgaagggcaatcagctgttgcccgtctcactggtgaaaagaaaaaccaccctggcgcccaatacgcaaaccgcctctccccgcgcgttggccgattcattaatgcagctggcacgacaggtttcccgactggaaagcgggcagtgagcgcaacgcaattaatgtaagttagcgcgaattgtcgaccaaagcggccatcgtgcctccccactcctgcagttcgggggcatggatgcgcggatagccgctgctggtttcctggatgccgacggatttgcactgccggtagaactccgcgaggtcgtccagcctcaggcagcagctgaaccaactcgcgaggggatcgagcccggggtgggcgaagaactccagcatgagatccccgcgctggaggatcatccagccggcgtcccggaaaacgattccgaagcccaacctttcatagaaggcggcggtggaatcgaaatctcgtgatggcaggttgggcgtcgcttggtcggtcatttcgaaccccagagtcccgctcagaagaactcgtcaagaaggcgatagaaggcgatgcgctgcgaatcgggagcggcgataccgtaaagcacgaggaagcggtcagcccattcgccgccaagctcttcagcaatatcacgggtagccaacgctatgtcctgatagcggtccgccacacccagccggccacagtcgatgaatccagaaaagcggccattttccaccatgatattcggcaagcaggcatcgccatgggtcacgacgagatcctcgccgtcgggcatgcgcgccttgagcctggcgaacagttcggctggcgcgagcccctgatgctcttcgtccagatcatcctgatcgacaagaccggcttccatccgagtacgtgctcgctcgatgcgatgtttcgcttggtggtcgaatgggcaggtagccggatcaagcgtatgcagccgccgcattgcatcagccatgatggatactttctcggcaggagcaaggtgagatgacaggagatcctgccccggcacttcgcccaatagcagccagtcccttcccgcttcagtgacaacgtcgagcacagctgcgcaaggaacgcccgtcgtggccagccacgatagccgcgctgcctcgtcctgcagttcattcagggcaccggacaggtcggtcttgacaaaaagaaccgggcgcccctgcgctgacagccggaacacggcggcatcagagcagccgattgtctgttgtgcccagtcatagccgaatagcctctccacccaagcggccggagaacctgcgtgcaatccatcttgttcaatcatgcgaaacgatcctcatcctgtctcttgatcagatcttgatcccctgcgccatcagatccttggcggcaagaaagccatccagtttactttgcagggcttcccaaccttaccagagggcgccccagctggcaattccggttcgcttgctgtccataaaaccgcccagtctagctatcgccatgtaagcccactgcaagctacctgctttctctttgcgcttgcgttttcccttgtccagatagcccagtagctgacattcatccggggtcagcaccgtttctgcggactggctttctacgtgttccgcttcctttagcagcccttgcgccctgagtgcttgcggcagcgtgaagcttaaaaaactgcaaaaaatagtttgacttgtgagcggataacaattaagatgtacccaattgtgagcggataacaatttcacacattaaagaggagaaattacatatg

SEQ ID NO: 9 - DNA (cDNA), 112 bp; both-stranded, both
aagcttaaaaaactgcaaaaaatagtttgacttgtgagcggataacaattaagatgtacccaattgtgagcggataacaatttcacacattaaagaggagaaattacatatg

SEQ ID NO: 10 - DNA (cDNA), 299 bp; both-stranded, both
aaaaatacagtggctttatttccattgtttatagtccccagtatcccatctgataagaaccttcaattctataaacaaaaatatttcaagaaagtatgttacacaatagtacatataagtaatagtttggcagaattttaaactctagtagttcatacccccaaaaaacaaattttaaanttcaaaaataacagttttatttaacatatgttacaccttaacatttaaaatatcatgctctagttaaatatttcatcaacaacactgtatacanntaaaatattacataaaatatattt

SEQ ID NO: 11 - DNA (cDNA), 282 bp; both-stranded, both
ttttgccagaaggcaagcaatccgggaatcctggggccaagaaagcaacgcagggaaccaaacggtggtgcgagtnttcctgctgggccagacacccccagaggacaaccaccccgacctttcagatatgctgaaatttgagagtgagaagcaccaagacattcttatgtggaactacagagacactttnttcaacttgtctctgaaggaagtgctgtttctnaggtgggtaagtacttcctgcccagacactgagtttgttttcaagggcgatgacgatgt

SEQ ID NO: 12 - DNA (cDNA), 266 bp; both-stranded, both
ttttgccagaaggcaagcaatccgggaatcctggggccaagaaagcaacgcagggaaccaaacggtggtgcgagtnttcctgctgggccagacacccccagaggacaaccaccccgacctttcagatatgctgaaatttgagagtnagaagcaccaagacattcttatgtggaactacagagacactttcttcaacttgtctctgaaggaagtgctgtttctcaggtgggtaagtacttcctgcccagacactgagtttgttttca

SEQ ID NO: 13 - DNA (cDNA), 361 bp; both-stranded, both
tttgcaaaatgagaatcatggtgtgacactcatctaatttatcttgttgtgatgttatggtcataataaggagaaanagggtttaattttncttgtatttggtttcctggtggtatcatagtgtaattttagtatttgaaaatcagtgtgattccttaatggccaactgaagattgaattgccgctaacaaccatatcgtgttagtgaatttncaatatggaccaggaaggcatatgtattttgaactcggagtgaaaaggttggaagttacagactttttggcataggtgggtttggtccaattttaaaattcccgaatttattnnttgncnnttnttncacatgggnggttattacagg

SEQ ID NO: 14 - DNA (cDNA), 259 bp; both-stranded, both
ttgttgggtatcctgatgatggcaaatgtcttcatttattttattatggaagtctccaaaagcagtagccaagaaaaaaatggaaaaggggaagtaataatacccaaagagaagttctggaagatatctacccctcccgaggcatactngaaccgagagcaagagaagctgaaccggcagtacaaccccatcctgagcatgctgaccaaccagacgggggaggcgggcaggctctccaatataagncatctgaactact

SEQ ID NO: 15 - DNA (cDNA), 195 bp; both-stranded, both
aaaaatacagtggctttatttccattgtttatagtccccagtatcccatctgataagaacctncaattctataaacaaaaatatttcaagaaagtatgttacacaatagtacatataagtaatagtttggcagaattttaaactctagtagttcatacccccaaaaaacaaattttaaaattcaaaaataacagt

SEQ ID NO: 16 - DNA (cDNA), 521 bp; both-stranded, both
ctataatattcctacttcccataataatgactgatttatttgtaattcaggtatttataaacctattggctacaaagactttgttaaacattatccagtggttttcgtgaaatggaattatgtttatttttatgggatttgggtaaattttaaattgtctagaaaactgaaatttcagttgtcagttgtggaattcagtttttcaattgtggaaatttcctgccaccccaacagtatttttgtgtgttaattaattttgcaaaatgagaatcatggtgtgacactcatctaatttatcttgttgtgatgttatggtcataatacggagaaagagggtttaatttttcttgtatttggtttcctggtggtatcatagtgtaattttagtatttgaaaatcagtgtgattccttaatggccaactgaagattgaattgccgctaacaaccatatcgtgttagtgaattttcaatatggaccaggaaggcatatgtaatttgaacttgagtgaaaaggttgaag

SEQ ID NO: 17 - DNA (cDNA), 517 bp; both-stranded, both
aatagatacaaactcaattttgcatagaaaggtatattttgaatagttcccatgttgtgttctcacattagagtaatttctgtattaaaccatgaaaattgcactttatgagtgatacccatttgagggcctctaaacccttcaatttggtactcacgtgaagagggaaagcggaagatggtaatttttttttacggatgatatggcaggatgattggttctgatcttaccggctagtggtcatttttaaaaaacttgtaccctcttatctgaaatcctgtttctggaatttggccattttaagtgattttgtttgccctcttctataatattcctacttcccataataatgactgatttatttgtaattcaggtatttataaacctattggctacaaagactttgttaaacattatccagtggttttcgtgaaatggaattatgtatatttttatgggatttgggaaattttaaattgtctagaaaactgaaatttcagttgtcagttgtggaatt

SEQ ID NO: 18 - DNA (cDNA), 462 bp; both-stranded, both
atactgttggggtggcaggaaatttccacaattgaaaaactgaattccacaactgacaactgaaatttcagttttctagacaatttaaaatttacccaaatcccataaaaataaacataattccatttcacgaaaaccactggataatgtttaacaaagtctttgtagccaataggtttataaatacctgaattacaaataaatcagtcattattatgggaagtaggaatattatagaagagggcaaacaaaatcacttaaaatggccaaattccagaaacaggatttcagataagagggtacaagttttttaaaaatngaccactagccggtaagatcagaaccaatcatcctgccatatcatccgtaaaaaaaaattaccatcttccgctttccctcttcacgtgagtaccaaattggaaggggttagaggccctcaaacgggtatcactcataaaggca

SEQ ID NO: 19 - DNA (cDNA), 448 bp; both-stranded, both
ctntaattaacacacaaaaatactgttggggtggcaggaaatttccacaattgaaaaactgaattccacaactgacaactgaaatttcagttttctagacaatttaaaatttacccaaatcccataaaaataaacataattccatttcacgaaaaccactggataatgtttaacaaagtctttgtagccaataggtttataaatacctgaattacaaataaatcagtcattattatgggaagtaggaatattatngaagagggcaaacaaaatcacttaaaatggccaaattccagaaacaggatttcagataagagggtacaagttttttaaaaatgaccactagccggtaagatcagaaccaatcatcctgccatatcatccgtaaaanaaaattaccatcttccgctttccctcttcacgtgagtaccaaattgaagggtttagg

SEQ ID NO: 20 - DNA (cDNA), 857 bp; both-stranded, both
ngnggtnncgtcggttaaatattcaagaccaaagccaaagatctcttcataggtgatgtgatccacaatgctggacctcatcgggataagaagctgaagtactacatcccagaagttgtttactctggcctctacccaccctatgcagggggaggggggttcctctactccggccacctggccctgaggctgtaccatatcactgaccaggtccatctctaccccattgatgacgtttatactggaatgtgccttcagaaactcggcctcgttccagagaaacacaaaggcttcaggacatttgatatcgaggagaaaaacaaaaataacatctgctcctatgtagatctgatgttaggacatagnaggaaaacctcaagagatgattgatatttgggctcaagntgcagagtgctcaatttaaaatgctaaaatagatacaaactcaatttgggattngaaggggtttttnggattggccccatntggggtcttnanattagagnnggttcaagtgggtacagtgatgaaaannnnnnnnnnnggnnnnnnnnccnnntnnttnnaannnnnnnnnnnnnnnnnnnnnnnntnncannnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnng

SEQ ID NO: 21 - DNA (cDNA), 467 bp; both-stranded, both
gagatgattgatatttggtctcagttgcagagtgctcatttaaaatgctaaaatagatacaaactcaattttgcatagaaaggtgtattttgaatagttcccatgttgtgttctcacattagagtaatttctgtattaaaccatgaaaattgcctttatgagtgatacccatttgagggcctctaaacccttcaatttggtactcacgtgaagagggaaagcggaagatggtaatttttttttacggatgatatggcaggatgattggttctgatcttaccggctagtggtcatttttaaaaaacttgtaccctcttatctgaaatcctgtttctgggaatttggccattttaagtgattttgtttgccctcttctatnaatattcctacttcccntaataatgactgatttnatttgtaantcaggnatttatnaaacccttgggctaccaagncttgttaaacat

SEQ ID NO: 22 - DNA (cDNA), 442 bp; both-stranded, both
tactgttggggtggcaggaaatttccaaaattgaaaaactgaattccacaactgacaactgaaatttcagttttctagacaatttaaaatttacccaaatcccataaaaataaacataattccatttcacgaaaaccactggataatgtttaacaaagtctttgtagccaataggtttataaatacctgaattacaaataaatcagtcattattatgggaagtaggaatattatagaagagggcaaacaaaatcacttaaaatggccaaattccagaaacaggatttcagataagagggtacaagttttttaaaaatgaccactagcccggtaagatcagaaccaatcatcctgccatatcatccgtaaaaaaaaattaccatcttccgctttccctcttcacgtgagtaccaaattggaaggggttagaggcccnccaacg

SEQ ID NO: 23 - DNA (cDNA), 575 bp; both-stranded, both
tactgttggggtcggcaggaaatttccacaattgaaaaactgaattccacaactgacaactgaaatttcagttttctagacaatttaaaatttacccaaatcccataaaaataaacataattccatttcacgaaaaccactggataatgtttaacaaagtctttgtagccaataggtttataaatacctgaattacaaataaatcagtcattattatgggaagtaggaatattatacgaagagggcaaacaaaatcacttaaaatggccaaattccagaaacaggatttcagataagagggtacaagttttttaaaaatgaccactagcccggtaagatcagaaccaatcatccctggccatatcatccggtaaaaaaaaattaccatcttccgcttttccctcttcacgtgaggtacccaattggaangggtttagaaggccctcaaacgggtatcactcnttaaaggcantttcatgggttaatatggaattaccnctaatggtgagaccccacctggggactattccaaataccccttccatggcaaattggngttggaaccantttagcat

SEQ ID NO: 24 - DNA (cDNA), 511 bp; both-stranded, both
ggatacccatttgagggcctctaaacccttcaatttggtactcacgtgaagagggaaagcggaagatggtaatttttttttatggatgatatggcaggatgattggttctgatcttaccggctagtggtcatttttaaaaaacttgtaccctcttatctgaaatcctgtttctggaatttggccattttaagtgattttgtttgccctcttctataatattcctacttcccataataatgactgatttatttgtaattcaggtatttataaacctattggctacaaagactttgttaaacattatccagtggttttcgtgaaatgggaattatgtttatttttatggggatttgggtaaattttaaattgtctaggaaaactgaaattttcagttgtccagttgtgggaattcagtttttccaattgtgggaaatttcccggccaccccaacagtatttttgtgtggttaattaatttttgccaaatgaggatccngggtgtgaccactnt

SEQ ID NO: 25 - DNA (cDNA), 400 bp; both-stranded, both
gtaaattttaaattgtctagaaaactgaaatttcagttgtcagttgtggaattcagtttttcaattgtggaaatttcctgccaccccaacagtatttttgtgtgttaattaattttgcaaaatgagaatcatggtgtgacactcatctaatttatcttgttgtgatgttatggtcataataaggagaaagagggtttaatttttcttgtatttggtttcctggtggtatcatagtgtaattttagtatttgaaaatcagtgtgattccttaatgggccaactgaagattgaattgccgctaacaaccatatcgtgttagtgaattttcaatatgggaccnggaagggcatatgtattttggaacttggagtggaaaaggttggagttacagacttttggc

SEQ ID NO: 26 - DNA (cDNA), 480 bp; both-stranded, both
tgagggtcacgtcggtggttacgggttttaacaacttgccggacagatttaaagactttctgctgtatttgagatgccgcaattattcactgcttatagatcagccggataagtgtgcaaagaaacctttcttgttgctggcgattaagtccctcactccacattttgccagaaggcaaggcaatccgggaatcctggggccaagaaagcaacgcagggaaccaaacggtggtgcgagtcttcctgctgggccagacacccccagaggacaaccaccccgacctttcagatatgctgaaattttgagagtgagaagcaccaagacattcttatgtgggaactacagaggacactttctttcaanttgtctntggaaggaagtgctgttttttcaggtggggttaagttatttcctgcccagacattgagtttgttttttcaaggggcgatggacgatgtttttgttgnacacccttcact

SEQ ID NO: 27 - DNA (cDNA), 392 bp; both-stranded, both
ttaattaacacacaaaaatactgttgggtanannaanaaatttccacaattgaaaaactgaattccacaactgacaactgaaatttcagttttctagacaatttaaaatttacccaaatcccataaaaataaacataattccatttcacgaaaaccactggataatgtttaacaaagtctttgtagccaataggtttataaatacctganttacaaataaatcagtcattattatgggaagtaggaatattatagaagagggcaaacaaantcacttaaaatggccaaattccagganacagggatttcagataagagggtacaagtnttttaaaagtgaccactaggccgggtaaggtccgganccaatcatcctgccatnttcatccgta

SEQ ID NO: 28 - DNA (cDNA), 381 bp; both-stranded, both
gnccccagtatcccatctgataagaaccttcaattctataaacaaaaatatttcaagaaagtatgttacacaatagtacatataagtaatagtttggcagaattttaaactctagtagttcatacccccaaaaaacaaattttaaaattcaaaaataacagttttatttaacatatgttacaccttaacatttaaaatatcatgctctagttaaatatttcatcaacaacactgtatacaantaaaatattacataanatatatttaaggnaaatgttttgggtctttgatctggaacantaaataaaaacacgggcacttctacataggacgggggtggcggttactactccaataataatcntggtntagggcggcctg

SEQ ID NO: 29 - DNA (cDNA), 323 bp; both-stranded, both
gatcaaagaccaaaacattttcttaaatatattttatgtaatattttatttgtatacagtgttgttgatgaaatatttaactagagcatgatattttaaatgttaaggtgtaacatatgttaaataaaactgttattttngaatttnaaaattngtttttngggggtatganctactagagtttaaaattctgccaaactattacttatatgtnctattgtgtaacatacttnctngaaatatttnggtttatagaattganggttcttatcagatgggatactggggactataaacaatggaaataaagccactgtatttnt

SEQ ID NO: 30 - DNA (cDNA), 299 bp; both-stranded, both; identical to SEQ ID NO: 10

SEQ ID NO: 31 - DNA (cDNA), 303 bp; both-stranded, both
gacagatttaaagactttctgctgtatttgagatgccgcaattattcactgcttatagatcagccggataagtgtgcaaagaaacctttcttgttgctggcgattaagtccctcactccacattttgccagaaggcaagcaatccgggaatcctggggccaagaaagcaacgcagggaaccaaacggtggtgcgagtcttcctgctgggccagacacccccagaggacaaccaccccgacctttcagatatgctgaaatttgagagttagaagcaccaagacattccttatgtgggacctaca

SEQ ID NO: 32 - DNA (cDNA), 317 bp; both-stranded, both
tttatttccattgtttatagtccccagtatcccatctgataagaaccttcaattctataaacaaaaatatttcaagaaagtatgttacacaatagtacatataagtaatagtttggcagaattttaaactctagtagttcatacccccaaaaaacaaattttaaaattcaaaaataacagttttatttaacatatgttacaccttaacatttaaaatatcatgctctagttaaatatttcatcaacaacactgtatacaaataaaatattacataaantatatttaaggnaaatgttttgggtctttgatctggaac

SEQ ID NO: 33 - DNA (cDNA), 325 bp; both-stranded, both
ttaaaaatanagtggctttatttccattgtttatagtccccagtatcccatctgataagaaccttcaattctataaacaaaaatatttcaagaaagtatgttacacaatagtacatataagtaatagtttggcagaattttaaactctagtagttcatacccccaaaaaacaaattttaaaattcaaaaataacagttttatttaacatatgttacaccttaacatttaaaatatcatgctctaggttaaatatttcatcaacaacactggtatacaaataaaatattacataaaatatatttaagggaaatgttttggggcttt

SEQ ID NO: 34 - DNA (cDNA), 282 bp; both-stranded, both; identical to SEQ ID NO: 11

SEQ ID NO: 35 - DNA (cDNA), 358 bp; both-stranded, both
aatttccacaattgaaaaactgaattccacaactgacaactgaaatttcagttttctagacaatttaaaatttacccaaatcccataaaaataaacataattccatttcacgaaaaccactggataatgtttaacaaagtctttgtagccaataggtttataaatacctgaattacaaataaatcagtcattattatgggaagtaggaatattatagaagagggcaaacaaaatcacttaaaatggccaaattccaggaaacagggatttcaggataagggggtacaagttttttaaaaatggaccactaggccgggtaaggatcaggaaccanttcatcctggccatattcatccgt

SEQ ID NO: 36 - DNA (cDNA), 428 bp; both-stranded, both
acttcctgcccagacactgagtttgttttcaagggcgatgacgatgtttttgtgaacacccatcacatcctgaattacttgaatagtttatccaagaccaaagccaaagatctcttcataggtgatgtgatccacaatgctggacctcatcgggataagaagctgaagtactacatcccagaagttgtttactctggcctctacccaccctatgcagggggaggggggttcctctactccggccacctgggcctgaggctgtaccatattcactggaccagggtccatctcttaccccattggatggacgttttatactgggaatgtgnccttcaggaaantcgggcctcgtttccaggaggaaacacaaagggtttcaggggacattttgatattcgaggggagggaaaaacaaaaanttaacattt

SEQ ID NO: 37 - DNA (cDNA), 266 bp; both-stranded, both; identical to SEQ ID NO: 12

SEQ ID NO: 38 - DNA (cDNA), 259 bp; both-stranded, both; identical to SEQ ID NO: 14

SEQ ID NO: 39 - DNA (cDNA), 247 bp; both-stranded, both
ttatagnccccagtatcccatctgataagaaccttcaattctataaacaaaaatatttcaagaaagtatgttacacaatagtacatataagnaatagtttggcagaattttaaactctagtagttcatacccccaaaaaacaaattttaaaattcaaaaataacagttttatttaacatatgttacaccttaacatttaaaatatcatgctctngttaaatatttcatcaacaacactgtatacaaa

SEQ ID NO: 40 - DNA (cDNA), 368 bp; both-stranded, both
ctgatgttagtacatagtagaaaacctcaagagatgattgatatttggtctcagttgcagagtgctcatttaaaatgctaaaatagatacaaactcaattttgcatagaaaggtgtattttgaatagttcccatgttgtgttctcacattagagtaatttctgtattaaaccatgaaaattgcctttatgagtgatacccatttgaggggcctcttaaacccttcaatttgggtacttcacgtgaagaggggaaagcgggaagatgggtaatttttttttacgggatggatatgggcngggatgattgggttctggatccttacccggcctagtgggtccattttttaaaaaacttggtacccccncc

SEQ ID NO: 41 - DNA (cDNA), 195 bp; both-stranded, both; identical to SEQ ID NO: 15

SEQ ID NO: 42 - DNA (cDNA), 2257 bp; both-stranded, both
gaaatatttttgtttatagaattgaaggttcttatcagatgggatactggggactataaacaatggaaataaagccactgtatttttaattttttgtgtaatgtgtaatctataatccttttgtttcccatatttgagaacatttttccctgaaagaggccagtttcctccccagaaaccattacagtagtgttgaactatcactgtctctcagtgcgtcatccatctttgcatttaaaatccccaaagtgctttcccatttaaagtctttaaagaaaagtgagaatatttatttatgcttccattttcagtgagtataaataatttaattagggagtggtgtggcattgtaaagattgtgttatcctaagccatttctattttggagtttgtagccacaaagatgaaatatagaatcagccttgactactcaatttcctttcatagacccatgttgagaagacactactaacgtccagtgggaaacaagtagacaattgatgaagctcaaaaaacagaagggttagtgttgtaagagcaaacagtctaatcctgtttggaatgtggaagccatttctgagcaagtatgaggacacaggtgcttgatttgagattgaagactgttttcagcctggtcttcctgaaggtttcctggggcctgcatctgccttctactcccatggctgctagcacacacctcccagagggccatattgccacattatggctagagaagagtaaagaagaaaagaagctctgagaacattcacaggtaattggatcacatttgcatttgtccaaaaaacctgaccacgcattctcaggtaataggtttctcctctcagaggaatttcaattttttttcttgttagagattccccttctctgaggtttcaagtctcttgtagagaaagaagagatggagcaggttttgaatgaggtgtggagggccactggggggccttttgtgagccttcagtccacatgtgtgctgttgtttgaacatgagttcttggtgctgatgacatttggatgagatgatctctggcccttcttcatttggcagaagttcttgtgcaatggctgcccaagcccaccacactggtcattgctgccctgtgagatggacctcatgggctttttagcagagcacgttaggttttagagctttacgcatgcttgggctctgttatggcgcaaacccttaaatccaggaaggcctctcttggtgcccacaatatgggttctcacctgatcccccatctcacggatggaactgctgtaagtctaacttattctttgagaactgtttaacaattaggcctcaagggaaactggtattttgggcccttttcttggctattcccaagtcatgttgattttgagtttgaaggtcaaaaaggctgaaagcattgccagggtttggactattcaaaaacccaagcaggtcttaaaaaaaggatgcaagagacaagaatggctcattccccttcctgatcctggttatacccatgtcctttcttgagatggtcaagagaggctggaaagaagaacaggaaattgggggagtgctttgttacacttggaaattgagtcaagaattaaagacacccaaagtgggccatctcctacttgtccacacctgattggtggtgatgcggaatatttgatgtcccgggtcatcttgactttctcagatgcaaaaagggagggtgactttactaatggaaaggatgggaagctgaaatgaatgaagccttcagttgggccaaagtttaacttccccgtgatttgccttctgatgaaaaatgccagatgaagtgaaaattcttgtttcttgcctagaacaggaaaatacatactttacatgctgggctattgaggctatgaaattaggttttccttaatgtaaatccaattgctagaaacatttgccaaataagattttttgaactgaactttgtttgcattaatctgaaaaactgaagtattctgactcatgaagttctcaaagtaatacactaaaaaagttttgcccttaataccattatatcttgtagaggccaagaatgagggacttctgtctttaaagagccctaaaaatctcgtttgctcacatgatatgaattaccgtatttgttgtaaatgcgcaactttgtatacactaaaagcactgccaatatgattttttatcagttgtgcctcagttagagatattaaaatgtgacatcttaaatattacatattagaataattg